(19) United States
(12) Patent Application Publication
(10) Pub. No.: US 2012/0232000 A1
(43) Pub. Date: Sep. 13, 2012

(54) METHODS AND COMPOSITIONS FOR PROMOTING REGENERATION BY INCREASING INTRACELLULAR SODIUM CONCENTRATION

(75) Inventor: Michael Levin, Swampscott, MA (US)

(73) Assignee: Tufts University, Boston, MA (US)

(21) Appl. No.: 13/387,869

(22) PCT Filed: Jul. 30, 2010
(86) PCT No.: PCT/US2010/043976

§ 371 Date: May 14, 2012

Related U.S. Application Data
(60) Provisional application No. 61/273,193, filed on Jul. 31, 2009.

(51) Int. Cl. A61K 38/28; A61K 31/35; C12N 5/0775; C12N 5/0735; C12N 5/071

(52) U.S. Cl. 514/5.9; 435/377; 435/325; 435/375; 514/460

(57) ABSTRACT

The invention provides methods and compositions for increasing the intracellular sodium concentration in a cell.

### RELATED APPLICATIONS

This application claims the benefit of priority to U.S. provisional application No. 61/273,193, filed Jul. 31, 2009, the disclosure of which is hereby incorporated by reference in its entirety.

### BACKGROUND OF THE INVENTION

Humans have limited ability to repair injured or damaged organs while newts and salamanders can regenerate multiple structures such as the heart and limbs (Birnbaum and Alvarado, 2008). A molecular understanding of the regenerative capacities of such species would greatly facilitate the development of therapies to repair human tissues upon disease or injury (Gardiner, 2005; Ingber and Levin, 2007). Much effort has recently gone into identifying and characterizing stem cells with the promise of therapeutics that may induce regeneration in humans (Stocum and Zupanc, 2008). In parallel, work has also been focused on understanding the natural molecular pathways that drive regeneration in animals that have such innate abilities (Slack, 2003; Yokoyama, 2008). One of these models is the anuran amphibian, Xenopus laevis, which has the ability to fully restore its developmental appendages upon injury (Beck et al., 2009; Tseng and Levin, 2008).

The Xenopus tail is a complex organ containing multiple cell types including muscle, nerve, spinal cord, and vasculature. It is transparent and easily accessible for experimentation. Notably, amputated tadpole tails will regenerate fully by 7 days. Appendage regeneration consists of three major steps. First, wound healing of the injury site occurs within 6-8 hours post amputation (hpa). By 24 hpa, an initial swelling is formed at the injury site called the regeneration bud, consisting of progenitor cells. Subsequently, tissue outgrowth and patterning begin as the tail is rebuilt. Lineage studies strongly indicate that each tissue is reconstituted from its own specific progenitor cells (Chen et al., 2006; Gargioli and Slack, 2004); no metaplasia has been observed in this system, suggesting its tissue renewal mechanisms are an attractive model for augmentation of organ repair in man. To date, several molecular components that regulate tail regeneration have been identified. TGF-beta signaling is required for proper wound healing (Ho and Whitman, 2008). Signaling pathways including BMP, Notch, Wnt, and Fgf are involved in driving regenerative outgrowth and patterning (Beck et al., 2006; Beck et al., 2003; Chen et al., 2006; Mochii et al., 2007), recapitulating their well-characterized roles during appendage development.

### SUMMARY OF THE INVENTION

Much work has gone into understanding mechanisms driving regeneration to develop biomedical interventions that induce or augment regeneration in human patients. The present disclosure stems from our novel approach to this problem; instead of focusing on secreted biochemical factors, we investigate the role of bioelectrical signals in regeneration. The strategy is to modulate the appropriate changes in ion flows to modulate regenerative response in a host tissue or organism. As part of that, the present invention provides methods and compositions for promoting tissue regeneration by increasing the intracellular sodium concentration. Also, the invention provides methods and compositions for inhibiting proliferation and/or migration of hyper-proliferative cells by decreasing the intracellular concentration of sodium in those hyper-proliferative cells.

In one aspect, the invention provides a method of promoting one or more of proliferation or differentiation, comprising contacting a cell culture with an effective amount of an agent to increase intracellular sodium concentration in cells of said cell culture, wherein said agent is selected from a sodium ionophore, or insulin, or both, and wherein the agent induces Na⁺ influx into said cell, thereby promoting one or more of proliferation or differentiation.

In another aspect, the invention provides a method of promoting tissue regeneration, comprising contacting a cell culture with an effective amount of an agent to increase intracellular sodium concentration in cells of said cell culture, wherein said agent is selected from a sodium ionophore, or insulin, or both, and wherein the agent induces Na⁺ influx into said cell, thereby promoting tissue regeneration.

In some aspects, the invention provides use of an agent for promoting one or more of proliferation or differentiation, comprising administering an effective amount of an agent, wherein said agent is selected from a sodium ionophore, or insulin, or both, and wherein the agent induces Na⁺ influx into cells, thereby promoting one or more of proliferation or differentiation.

In other aspects, the invention provides use of an agent for promoting tissue regeneration, comprising administering an effective amount of an agent, wherein said agent is selected from a sodium ionophore, or insulin, or both, and wherein the agent induces Na⁺ influx into said cells, thereby promoting tissue regeneration.

In certain embodiments of any of the foregoing, said sodium ionophore is monensin. In related embodiments, said Na⁺ influx does not alter the membrane potential of said cells. In some embodiments, any of the foregoing method or use promotes regeneration of an appendage or organ. In other embodiments, any of the foregoing method or use promotes regeneration of one or more of muscle tissue and neuronal tissue.

In certain embodiments of any of the foregoing, the cells comprise progenitor cells. In certain embodiments, the cells are a culture of progenitor cells, such as a culture of substantially purified progenitor cells. In some embodiments, said progenitor cell is selected from one or more of an embryonic stem cell, a neural progenitor cell, a neural crest progenitor cell, a mesenchymal stem cell, or a muscle progenitor cell.

In some embodiments of any of the foregoing, prior to contact with said agent, the cell culture or external milieu comprises a medium having a higher sodium concentration relative to the intracellular sodium concentration of the cell. In certain embodiments, prior to contact with said agent, the cells are in a non-proliferative state.

In one embodiment of any of the foregoing, said agent induces Na⁺ influx into said cells via an endogenously expressed voltage-gated sodium channel.

In another aspect, the invention provides use of an agent selected from one or more of an ionophore or a sodium channel modulator that promote sodium efflux for inhibiting growth and/or metastasis of tumor cells.

In one embodiment of any of the foregoing, said Na⁺ efflux does not alter the membrane potential of said cell. In certain embodiments, said method inhibits migration and metastasis of the tumor cell.

In one aspect, the invention provides a method of promoting one or more of proliferation or differentiation, comprising administering an amount of an agent effective to increase intracellular sodium concentration in a cell, wherein said agent induces Na⁺ influx into said cell, thereby promoting one or more of proliferation or differentiation.

For example, using such a method, proliferation or differentiation of the same cell into which Na+ influx is induced is promoted.

In another aspect, the invention provides a method of promoting tissue regeneration, comprising administering an amount of an agent effective to increase intracellular sodium concentration in a cell in a tissue, wherein said agent induces Na⁺ influx into said cell, thereby promoting cell proliferation to promote tissue regeneration. In certain embodiments, the method promotes innervation of the tissue.

In certain embodiments of any of the foregoing, the agent induces Na⁺ influx into said cell via an endogenously expressed voltage-gated sodium channel, e.g., a Na_{V}1.2 channel, Na_{V}1.5 channel, ENaC channel. In other embodiments, the voltage-gated sodium channel may be introduced into the cell exogenously (e.g., by transfection or electroporation). In a related embodiment, the agent is a voltage-gated sodium channel opener, such as an Na_{V}1.2 channel opener. In some embodiments, the agent is a sodium ionophore, e.g., monensin, Gramicidin A. In other embodiments, the agent that induces Na⁺ influx into said cell is insulin. In any of the foregoing aspects and/or embodiments, the method further comprises administering said agent in the presence of a medium having a higher sodium concentration relative to the intracellular sodium concentration in the cell prior to administration of said agent. In other embodiments, said Na⁺ influx does not alter the membrane potential of said cell. In some embodiments, said cell is in a non-regenerative state prior to administration of said agent.

In any of the foregoing aspects and/or embodiments, said method promotes regeneration of an appendage, or an organ, or regeneration of one or more of muscle tissue and neuronal tissue. In certain embodiments, the cell is a mesenchymal cell. In any of the foregoing aspects and/or embodiments, the cell is a progenitor cell, selected from one or more of an embryonic stem cell, a neural progenitor cell, a neural crest progenitor cell, a mesenchymal stem cell, or a muscle progenitor cell. In some embodiments, the method comprises administering said agent to a culture comprising said progenitor cell.

In any of the foregoing embodiments, the agent can be a small molecule, such as a small organic molecule.

In certain aspects, the invention provides a method of promoting tissue regeneration of a tissue comprising cells in a non-proliferative state. In other words, the tissue is in a non-regenerative state. The method comprises, administering an amount of an agent effective to increase intracellular sodium concentration in a cell in said tissue, wherein said agent induces Na⁺ influx into said cell, thereby promoting cell proliferation to promote regeneration. For example, using such a method, cell proliferation of the same cell into which Na⁺ influx is induced is promoted.

In other aspects, the invention also provides a method of promoting one or more of proliferation or differentiation of a cell that is in a non-proliferative state, comprising administering an amount of an agent effective to increase intracellular sodium concentration in a cell, wherein said agent induces Na⁺ influx into said cell, thereby promoting one or more of proliferation or differentiation.

In certain embodiments of any of the foregoing, the method promotes innervation of said tissue.

In certain embodiments of any of the foregoing, the agent induces Na⁺ influx into said cell via an endogenously expressed voltage-gated sodium channel, e.g., a Na_{V}1.2 channel, Na_{V}1.5 channel, ENaC channel. In other embodiments, the voltage-gated sodium channel may be introduced into the cell exogenously (e.g., transfected or electroporated). In a related embodiment, the agent is a voltage-gated sodium channel opener, such as an Na_{V}1.2 channel opener. In some embodiments, the agent is a sodium ionophore, e.g., monensin, Gramicidin A.

In other embodiments, the agent that induces Na⁺ influx into said cell is insulin. In any of the foregoing aspects and/or embodiments, the method further comprises administering said agent in the presence of a medium having a higher sodium concentration relative to the intracellular sodium concentration in the cell prior to administration of said agent. In other embodiments, said Na⁺ influx does not alter the membrane potential of said cell.

In any of the foregoing aspects and/or embodiments, said method promotes regeneration, in whole or in part, of an organ, an appendage, or regeneration of one or more of muscle tissue and neuronal tissue. In certain embodiments, the cell is a mesenchymal cell.

In any of the foregoing embodiments, the agent can be a small molecule.

Another aspect of the invention provides a method for determining whether cells in a sample are in a wound healing state or in a regenerative state, comprising contacting said sample with a compound that detects expression of a Na_{V}1.2 channel, wherein a sample in which cells express the Na_{V}1.2 channel are identified as being in a regenerative state rather than in a wound healing state. In some embodiments, said sample is a tissue, such as an organ, for which regeneration is desired. By way of example, in certain embodiments, said sample comprises the blastema of an amputated appendage.

In certain embodiments, the method comprises detecting mRNA or protein expression of the Na_{V}1.2 channel. In some embodiments, the compound is an antisense probe that hybridizes to a nucleic acid encoding the Na_{V}1.2 channel. In other embodiments, the compound is an antibody that binds specifically to the Na_{V}1.2 channel.

In other aspects, the invention provides a method of upregulating expression in a cell of one or more genes that promote tissue regeneration, comprising administering an amount of an agent effective to increase intracellular sodium concentration in a cell, wherein said agent induces Na⁺ influx into said cell, thereby upregulating expression in said cell of one or more genes that promote tissue regeneration.

In another aspect, the invention also provides a method of upregulating expression in a cell of one or more genes that promote proliferation or differentiation, comprising administering an amount of an agent effective to increase intracellular sodium concentration in a cell, wherein said agent induces Na⁺ influx into said cell, thereby upregulating expression in said cell of one or more genes that promote proliferation or differentiation.

In any of the foregoing aspects, examples of genes that promote proliferation, differentiation, and/or regeneration include Notch and MSX1. In certain embodiments, expression of such genes is evaluated using compounds that detect mRNA or protein expression of such genes.

In certain embodiments, the agent induces Na⁺ influx into said cell via an endogenously expressed voltage-gated sodium channel, e.g., a Na_{V}1.2 channel, Na_{V}1.5 channel, ENaC channel. In other embodiments, the voltage-gated sodium channel may be introduced into the cell exogenously (i.e., transfected or electroporated). In a related embodiment, the agent is a voltage-gated sodium channel opener, such as an Na_{V}1.2 channel opener. In some embodiments, the agent is a sodium ionophore, e.g., monensin, Gramicidin A. In other embodiments, the agent that induces Na⁺ influx into said cell is insulin. In any of the foregoing aspects and/or embodiments, the method further comprises administering said agent in the presence of a medium having a higher sodium concentration relative to the intracellular sodium concentration in the cell prior to administration of said agent. In other embodiments, said Na⁺ influx does not alter the membrane potential of said cell. In some embodiments, said cell is in a non-proliferative state prior to administration of said agent.

In certain embodiments of any of the foregoing aspects and/or embodiments, said method promotes regeneration of an appendage, an organ, or regeneration of one or more of muscle tissue and neuronal tissue. In certain embodiments, the cell is a mesenchymal cell. In any of the foregoing aspects and/or embodiments, the cell is a progenitor cell, selected from one or more of an embryonic stem cell, a neural progenitor cell, a neural crest progenitor cell, a mesenchymal stem cell, or a muscle progenitor cell. In some embodiments, the method comprises administering said agent to a culture comprising said progenitor cell.

In certain embodiments of any of the foregoing aspects and/or embodiments, the agent can be a small molecule.

In certain embodiments of any of the foregoing aspects and/or embodiments, the cell into which an increase in intracellular sodium concentration is promoted does not express an exogenously introduced nucleic acid encoding a voltage-gated sodium channel. In other embodiments, the cell into which an increase in intracellular sodium concentration is promoted does not express any exogenously introduced nucleic acid encoding an ion transporter.

In certain embodiments of any of the foregoing aspects and/or embodiments, the method does not appreciably alter the membrane potential of the cell in which intracellular sodium concentration is increased.

In another aspect, the disclosure provides a method for inhibiting one or more of proliferation or migration of a hyper-proliferative cell. The method comprises administering an amount of an agent effective to decrease intracellular sodium concentration in said hyperproliferative cell. The agent decreases intracellular sodium concentration by, for example, inducing Na⁺ efflux from said cell or preventing further sodium influx into said cell, thereby inhibiting one or more of proliferation or migration of said hyper-proliferative cell.

In a related aspect, the disclosure provides a method of inhibiting growth and/or metastasis of a tumor. The method comprises administering an amount of an agent effective to decrease intracellular sodium concentration in cells in said tumor. The agent decreases intracellular sodium concentration by, for example, inducing Na⁺ efflux from said cell or preventing further sodium influx into said cell, thereby inhibiting cell proliferation to inhibit growth and/or metastasis of the tumor.

In certain embodiments of the foregoing, the agent induces Na+ efflux from said cell via an endogenously expressed voltage-gated sodium channel. In certain embodiments, the method further comprises administering said agent in the presence of a medium having a lower sodium concentration (external to the cell) relative to the intracellular sodium concentration in the cell prior to administration of said agent. In certain embodiments, the agent closes or disrupts a voltage-gated sodium channel to prevent further sodium influx into said cells. In some embodiments, the agent may be a small molecule, or a nucleic acid (e.g., antisense, RNAi). In a related embodiment, the agent may also be a polypeptide (e.g., a dominant negative variant of a hyperactive voltage-gated sodium channel).

In certain embodiments of any of the foregoing or following, the decrease in intracellular sodium concentration does not alter the membrane potential of said cell. In certain embodiments, the cell in which a decrease in intracellular sodium concentration is promoted does not express an exogenously introduced nucleic acid encoding a sodium channel. In certain embodiments, the cell in which a decrease in intracellular sodium concentration is promoted does not express any exogenously introduced nucleic acid encoding an ion transporter. In certain embodiments, the cell in which a decrease in intracellular sodium concentration is promoted does not endogenously express a voltage-gated sodium channel. In certain embodiments, the cell in which a decrease in intracellular sodium concentration is promoted does endogenously express a voltage-gated sodium channel, and the method comprises administering an agent that disrupts the channel by inhibiting expression and/or activity of the channel.

In certain embodiments of any of the foregoing, the method inhibits growth of a tumor comprising cells in which the intracellular sodium concentration is modulated. In certain embodiments, the method inhibits migration and metastasis of a tumor comprising said cells.

In certain embodiments of any of the foregoing, the cells in which sodium concentration is modulated are tumor stem cells.

In certain embodiments of any of the foregoing, the method comprises administering said agent to a culture comprising said cell.

In certain embodiments of any of the foregoing, the agent is a small molecule.

The invention contemplates combinations of any of the foregoing or following aspects and embodiments of the invention.

### BRIEF DESCRIPTION OF THE DRAWINGS

FIG. 1. Na_{V}1.2 function is required endogenously for normal tail regeneration. Immunohistochemistry of wholemount amputated tails using an anti-Na_{V}1.2 antibody. (A) A 12 hpa regeneration bud showing absence of Na_{V}1.2 protein. (B) By 24 hpa, Na_{V}1.2 protein strongly detected in the regeneration bud. (C) At 48 hpa, individual cells in the new tissue express Na_{V}1.2. (D) A section through the regeneration bud showing that Na_{V}1.2 is specifically expressed in the mesenchymal cells but not in the wound epidermis. Red arrows indicate expression; white arrows indicate lack of expression. (E) V-ATPase (purple staining) is expressed at the wound edge at 24 hours post injury. (F) In contrast, Na_{V}1.2 is absent from the wound edge at the same timepoint. (G-I) A st. 40 animal injected at the 1-cell stage with Na_{V}1.2 hairpin RNAi construct that also expresses GFP. Yellow arrows indicate the amputation plane. (G) Brightfield image of the tail expressing RNAi construct immediately after amputation. (H) Expression of GFP in the same tail as in panel G. (I) Comparison of the effects of Na_{V}1.2 or dsRED RNAi in the tail reveals specific inhibition of regeneration by loss of Na_{V}1.2. In all figures, dorsal is up and anterior is to the left. Scale bar=500 μm except for (D) bar=100 μm.

FIG. 2. Na_{V}1.2 functions during the first 48 hours post amputation. (A) Control tails amputated at st. 40 regenerate fully after 7 days, (B) while siblings treated with 250 μM MS-222 failed to regenerate. Yellow arrows show the amputation plane. (C) Quantitative comparison of phenotypes from 2 different concentrations of MS-222 treatment as compared to controls showing that the inhibition of regeneration is dosage-dependent. (D) Histogram showing the inhibition of regeneration (expressed as % of animals with poor or no regenerate tissue for each treatment as compared to controls) under different treatment regimes. Treatment with 250 μM MS-222 demonstrated that about half of the inhibitory activity takes places during the first 24 hpa, and a 0-48 hpa exposure is sufficient to exert maximal inhibition. Notably, exposure initiated past 24 hpa is much less effective, revealing the early requirement of Na_{V}1.2 function during regeneration. Scale bar=1 mm. * indicates p<0.05 as compared to 0-7 dpa treatment.

FIG. 3. Inhibition of Na_{V}1.2 activity blocks Na⁺ transport and results in regenerative failure. (A-C) Signal from CoroNa Green (green), a fluorescent indicator dye of Na⁺ flux (red arrows). (A) In uncut tails, only scattered cells exhibit CoroNa Green signal. (B) Under normal conditions, Na⁺ influx into the regeneration bud (outlined by white circles) is seen at 24 hpa. (C) Exposure to MS-222 blocks Na⁺ entry into the bud. A quantitative comparison of the signal intensities between normal and MS-222-treated buds showed that there is a 70% decrease in signal in Na_{V}1.2-inhibited buds as compared to control buds (p<0.02; data not shown). (D) Histogram (right panel) showing a significant reduction of the number of H3P-positive cells at 48 hpa in regeneration buds treated with MS-222 as compared to wild-type. In contrast, the number of H3P-positive cells in the central tail region is comparable to wild-type at 48 hpa (left panel), suggesting that the proliferation defect is a regeneration-specific, spatially-localized effect. Bars indicate standard deviation. (E-F) Immunohistochemistry of 48 hpa tails identifying mitotic cells using an anti-H3P antibody (blue signal, yellow arrows in E and F). (E) Sagittal sections of an amputated control tail show many mitotic cells in the bud. (F) Buds of tadpoles exposed to MS-222 have few mitotic cells. (G-H) 72 hpa tails stained with an antibody to acetylated α-tubulin to identify axons. (G) In controls, axon bundles run parallel to the A-P axis and concentrate at the tip of the regenerate (yellow arrow). (H) In Na_{V}1.2-inhibited tails, axons are reduced in numbers (white arrow) and trace along the edge of the amputation site. (A-F) Scale bar=500 μm. (G-H) Scale bar=50 μm.

FIG. 4. NaV acts downstream of V-ATPase to regulate expression of genes involved in regenerative outgrowth. (A) Na_{V}1.2 is expressed in the 24 hpa regeneration bud (red arrow) of amputated tails. In contrast, Na_{V}1.2 protein is absent in 24 hpa (B) and 48 hpa (C) tails stumps treated with the V-ATPase inhibitor, Concanamycin (white arrow shows absence of expression). (D) In 24 hpa tails cut during the non-regenerative refractory stage, Na_{V}1.2 protein is also not expressed. (E) Treatment of regeneration buds with the depolarizing reagent Palytoxin also abolishes Na_{V}1.2 expression, suggesting that Na_{V}1.2 expression is regulated by the membrane potential state of the regenerate. (F-M) RNA in situ hybridizations at 48 hpa of Notch and Msx1. Panels F, H, J, and L are control tails whereas panels G, I, K, and M are tails treated with MS-222 after amputation. (F) Notch RNA is normally expressed in the neural ampulla (red arrow) and in the mesenchyme of the regeneration bud but is greatly down-regulated and mis-localized when Na_{V}1.2 activity is blocked (black arrow) (G). (J) Msx1 is expressed in the amputation edge and the neural ampulla (red arrow) of the regenerating appendage. In the presence of MS-222, Msx1 RNA is absent (black arrow) (K). (H, I, L, and M) are sagittal sections through the regenerate. Scale bar=250 μm. (O-P) Comparison of the relative voltage patterns of tail regeneration buds at 24 hpa using the voltage dye, DiBAC₄(3). Green is more depolarized than blue. Distal tail end is outlined in white. Scale bar=100 μm. (O) The regeneration bud (red circle) of controls was polarized (blue color). (P) MS-222 treated buds show a similar pattern. (Q) Quantitative comparison showing the strong reduction in tail regenerative ability of animals treated with the SIK inhibitor, Staurosporine, as compared to controls (p<0.001, n=52).

FIG. 5. Short term induction of Na⁺ current is sufficient to induce regeneration. (A-B) Comparison of wound epidermis at 18 hpa (red arrows bracketing WE). (A) Wound healing in refractory buds results in a thickened epidermis by 18 hpa. (A′) The width of epidermis is outlined by a dashed red line, 2.5 pt. (B) This thick epidermis is not observed in regeneration buds that are competent to fully restore the tail. (B′) The epidermis width outlined by a dashed red line, 1.25 pt. (C-G) Tails of refractory stage tadpoles were amputated. At 18 hpa, tadpoles were treated with or without 90 mM Na⁺ and 20 μM monensin for 1 hour, and transferred back to normal culture medium. Yellow arrows indicate the amputation plane. (C) A CoroNa Green analysis of refractory tails showed that non-regenerative buds contain low level of intracellular Na⁺ (green signal) at 19 hpa. (D) In contrast, tails treated with monensin and high extracellular Na⁺ stimulated Na⁺ transport into the bud resulting in a significantly stronger CoroNa Green signal in the amputated tail. Images are shown as a merge of the brightfield image and CoroNa Green fluorescence of the same exposure time. (E) At 7 dpa, amputated of the refractory period regenerate poorly. (F) A transient Na⁺ current was able to induce full regeneration. (G) In controls, 11% of tadpoles have poor regenerates while the majority failed to regenerate. Stimulation of Na⁺ current with monensin specifically induced regeneration in 36% of the tadpoles, representing a 3-fold increase over the control. Importantly, regeneration quality was greatly improved as 45% of these tails show good to full regeneration. Scale bar=(A-B) 250 μm; (C—F) 500 μm.

FIG. 6. A model for integrating Na_{V}1.2 in caudal regeneration. By 6 hpa, the H⁺ pump, V-ATPase is expressed in the regeneration bud and is required to regulate the membrane voltage of the bud. The activation of V-ATPase in turn results in the up-regulation of Na_{V}1.2 by 18 hpa. Ablation of Na_{V}1.2 expression (RNAi) or activity (pharmacological treatment) inhibits regeneration. Na_{V}1.2 activity enables sodium ions to enter the cells of the regeneration bud. Moreover, Na_{V}1.2 functions in the regeneration bud to modulate downstream pathways (such as BMP and Notch) that are activated by 24 hpa to drive regenerative outgrowth and patterning. By 7 days after injury, the rebuilding of the tail is largely complete. Importantly, monensin-mediated induction of a transient sodium flux into non-regenerative buds is sufficient to restore full tail regeneration, demonstrating that intracellular sodium signaling is a key regulator of regeneration that can initiate the repair even after a non-regenerative wound epithelium has formed.

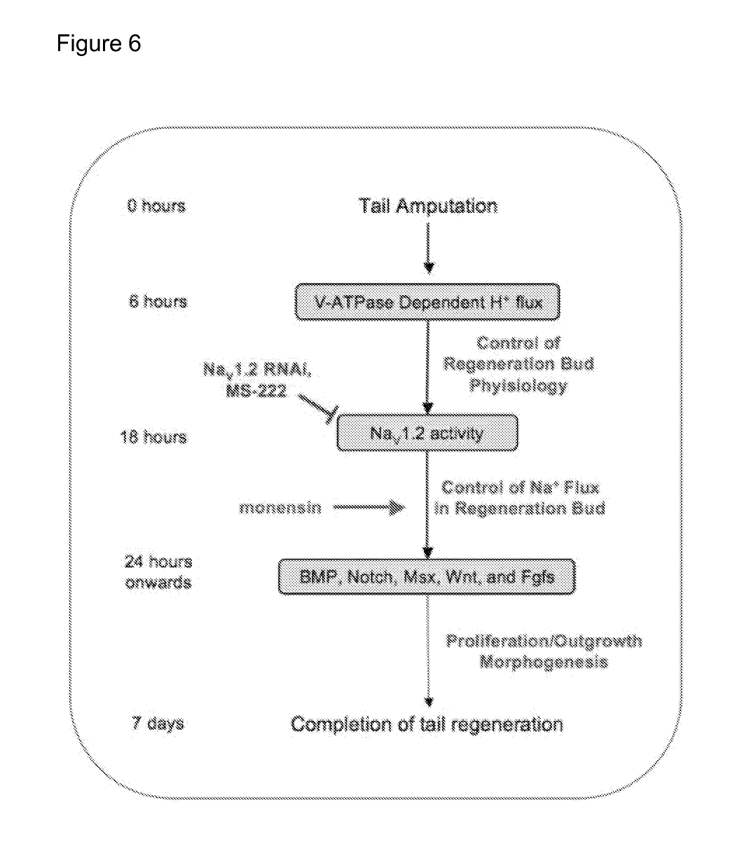

FIG. 7. Detailed role of Na_{V}1.2 in endogenous regeneration in the Xenopus tail. After tail amputation, V-ATPase-mediated repolarization of the bud cells upregulates expression of Na_{V}1.2 by 18 hpa. Na_{V}1.2 enables Na⁺ ions to enter the regeneration bud cells, leading (perhaps through the HDAC kinase SIK1) to the activation of downstream regenerative signaling pathways (induction of proliferation and axonal guidance). By 7 dpa, the rebuilding of the tail is complete. In contrast, tails amputated during the refractory period remain depolarized and form a non-functional wound epidermis by 18 hpa, likely blocking regeneration. Induction of a transient Na⁺ flux into non-regenerative refractory buds at 18 hpa provides the necessary activating signal to proceed with regeneration, demonstrating that intracellular Na⁺ signaling is a key regulator of regeneration that can initiate repair even after a non-regenerative wound epithelium has formed.

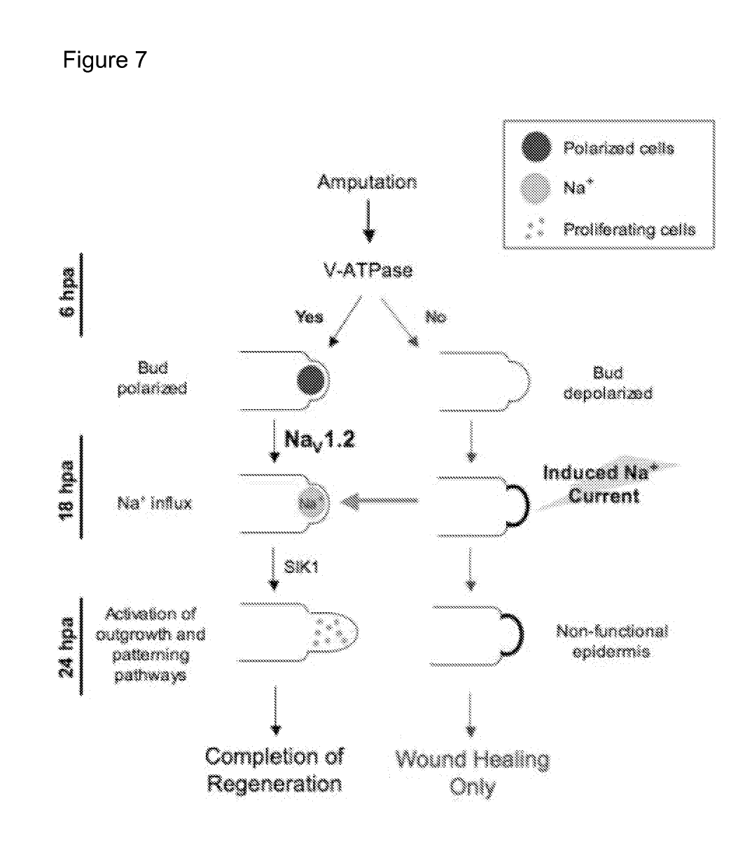

FIG. 8. Treatment with insulin after amputation rescues tail regeneration during the refractory period. Tadpole tails amputated during the refractory stage were treated with insulin at 0 hours post amputation. Tail regeneration was assayed at 7 days post amputation. Control tails grew poorly with 84% of animals showing either poor or no regeneration, and only 16% showing good or full regeneration (Regeneration Index=62, with RI of 300 indicating full regeneration for all animals). Treatment with insulin significantly increased regenerative ability, increasing the number of animals showing good or full regeneration to 39% (RI=118, total N-238; U-9510, T<0.001).

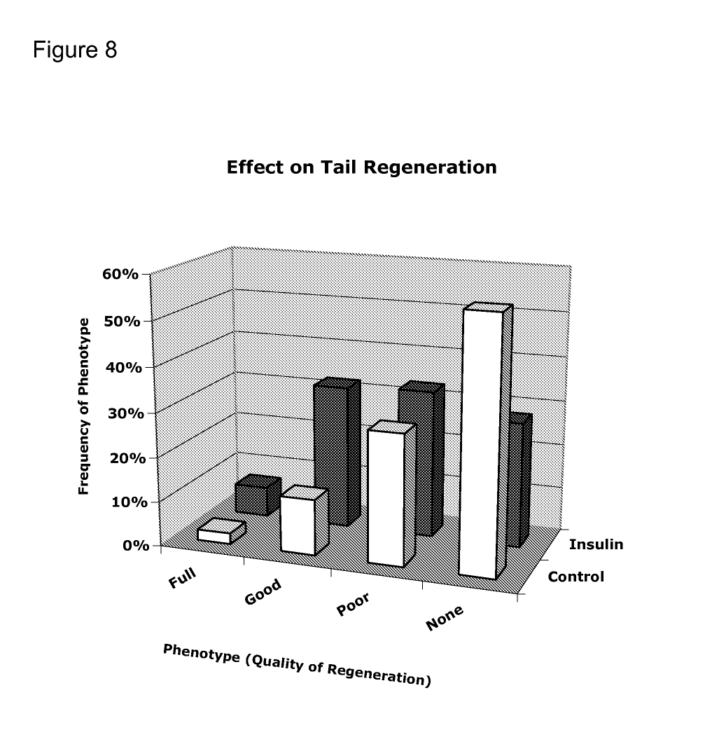

### DETAILED DESCRIPTION OF THE INVENTION

### I. Overview

Understanding regeneration is essential for biomedicine. The present invention discloses, in part, a novel role for intracellular sodium concentration in vertebrate proliferation and regeneration.

The experiments summarized herein indicate that during endogenous regeneration of the Xenopus tail, the voltage-gated sodium channel, Na_{V}1.2, mediates changes in intracellular sodium concentration important for proper regeneration. Briefly, this channel becomes expressed in the amputated Xenopus tail bud within 18 hours post amputation (hpa) and produces an increase in intracellular sodium in the bud after injury. Inhibition of Na_{V} abolishes sodium influx, causing regenerative failure. Without being bound by theory, the sodium influx appears necessary to induce expression of downstream genes required for tail outgrowth and patterning, and thus required for a regenerative response in the injured tissue.

Moreover, Na_{V}1.2 is an endogenous component of the regenerative response in the tail and is absent under non-regenerative conditions (e.g., during the refractory period). However, despite the lack of expression of this channel during non-regenerative periods, artificial induction of sodium influx into cells of the regeneration bud at 18 hpa (for example, using a sodium ionophore that promotes sodium influx in a channel-independent fashion) is sufficient to restore regeneration of the tail during non-regenerative stages. Thus, although Na_{V}1.2 may endogenously regulate sodium flux during tissue regeneration in the Xenopus tail, the channel itself is not required for regeneration. Rather, it is the increase in intracellular sodium concentration that it important for promoting proliferation and regeneration, and modulating Na_{V}1.2 activity represents one of many mechanisms for increasing intracellular sodium concentration.

Additionally, these experiments during the refractory period demonstrate that non-regenerative wound repair is not a permanent block to regeneration. This suggests that scarring and other non-regenerative responses to injury can be overcome and do not represent permanent blocks to productive tissue regeneration.

Furthermore, the present disclosure also provides methods of modulating Na+ flux to inhibit one or more of proliferation and/or migration of a hyper-proliferative tissue, or cells derived from such tissue. In a related embodiment, the present disclosure provides a method for inhibiting growth and/or metastasis of a tumor.

The present invention shows that sodium transport provides a mechanism for controlling regeneration, and demonstrates that modulation of sodium transport represents a new approach for promoting tissue repair and/or for modulating stem cell behavior. Given this evidence of the role of sodium concentration in promoting regeneration, the present disclosure provides numerous methods and reagents for increasing or decreasing sodium concentration in a cell.

### II. Definitions

The articles “a” and “an” are used herein to refer to one or to more than one (i.e., to at least one) of the grammatical object of the article. By way of example, “an element” means one element or more than one element.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The term “wild type” refers to the naturally-occurring polynucleotide sequence encoding a protein, or a portion thereof, or protein sequence, or portion thereof, respectively, as it normally exists in vivo. The term “wild type” also refers to a phenotypically and genotypically normal organism.

The term “mutant” refers to any change in the genetic material of an organism, in particular a change (i.e., deletion, substitution, addition, or alteration) in a wildtype polynucleotide sequence or any change in a wildtype protein sequence. The term “variant” is used interchangeably with “mutant”. Although it is often assumed that a change in the genetic material results in a change of the function of the protein, the terms “mutant” and “variant” refer to a change in the sequence of a wildtype protein regardless of whether that change alters the function of the protein (e.g., increases, decreases, imparts a new function), or whether that change has no effect on the function of the protein (e.g., the mutation or variation is silent). The term “mutant” also refers to an organism with one or more phenotypic or genotypic alterations in comparison to a wild type organism of the same species.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

The terms “compound,” “modulator” and “agent” are used interchangeably to refer to nucleic acids, peptides, polypeptides, or small molecules. In the context of the present invention, compounds or agents may modulate ion flux, for example, by inhibiting or promoting ion flux mediated by a particular ion transporter protein or class of ion transporter proteins. Further, compounds or agents include, for example, ionophores, which do not require a particular ion transporter protein. Additionally, as demonstrated herein, certain proteins, e.g., insulin, can also be used to promote sodium influx. Exemplary nucleic acid agents include, but are not limited to, sense or antisense nucleic acids, sense or antisense oligonucleotides, ribozymes, and RNAi constructs. Exemplary peptide and polypeptide agents include growth factors, transcription factors, peptidomimetics, and antibodies, as well as particular ion transporter proteins or subunits thereof. Exemplary small molecules include small organic or inorganic molecules, e.g., with molecular weights less than 5000 amu, and even more preferably less than 2000, 1500, 1000, or 500 amu.

A “marker” is used to determine the state of a cell. Markers are characteristics, whether morphological or biochemical (enzymatic), particular to a cell type, or molecules expressed by the cell type. A marker may be a protein marker, such as a protein marker possessing an epitope for antibodies or other binding molecules available in the art. A marker may also consist of any molecule found in a cell, including, but not limited to, proteins (peptides and polypeptides), lipids, polysaccharides, nucleic acids and steroids. Additionally, a marker may comprise a morphological or functional characteristic of a cell. Examples of morphological traits include, but are not limited to, shape, size, and nuclear to cytoplasmic ratio. Examples of functional traits include, but are not limited to, the ability to adhere to particular substrates, ability to incorporate or exclude particular dyes, ability to migrate under particular conditions, the ability to differentiate along particular lineages, and the ability to restore (i.e., regenerate) all or a portion of a tissue or an organ (e.g., an appendage).

Markers may be detected by any method available to one of skill in the art. In addition to antibodies (and all antibody derivatives) that recognize and bind at least one epitope on a marker molecule, markers may be detected using analytical techniques, such as by protein dot blots, sodium dodecyl sulfate polyacrylamide gel electrophoresis (SDS-PAGE), or any other gel system that separates proteins, with subsequent visualization of the marker (such as Western blots), gel filtration, affinity column purification; morphologically, such as fluorescent-activated cell sorting (FACS), staining with dyes that have a specific reaction with a marker molecule (such as ruthenium red and extracellular matrix molecules), specific morphological characteristics (such as the presence of microvilli in epithelia, or the pseudopodia/filopodia in migrating cells, such as fibroblasts and mesenchyme); and biochemically, such as assaying for an enzymatic product or intermediate, or the overall composition of a cell, such as the ratio of protein to lipid, or lipid to sugar, or even the ratio of two specific lipids to each other, or polysaccharides. In the case of nucleic acid markers, any known method may be used. If such a marker is a nucleic acid, PCR, RT-PCR, in situ hybridization, dot blot hybridization, Northern blots, Southern blots and the like may be used, coupled with suitable detection methods. If such a marker is a morphological and/or functional trait, suitable methods include visual inspection using, for example, the unaided eye, a stereomicroscope, a dissecting microscope, a confocal microscope, or an electron microscope.

“Differentiation” describes the acquisition or possession of one or more characteristics or functions different from that of the original cell type. A differentiated cell is one that has a different character or function from the surrounding structures or from the precursor of that cell (even the same cell). The process of differentiation gives rise from a limited set of cells (for example, in vertebrates, the three germ layers of the embryo: ectoderm, mesoderm and endoderm) to cellular diversity, creating all of the many specialized cell types that comprise an individual.

Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway. In many, but not all tissues, the process of differentiation is coupled with exit from the cell cycle. In these cases, the cells typically lose or greatly restrict their capacity to proliferate and such cells are commonly referred to as being terminally differentiated.

The term regeneration refers to the restoration of cells, tissues, organs, or structures (e.g., an appendage) following injury, ablation, loss, or disease. Regeneration involves an interplay of proliferation, differentiation, sometimes dedifferentiation, innervation, and migration of cells, alone or in any combination, depending on the particular tissue. In some instances, regeneration refers to individual cells or groups of cells. In other instances, regeneration comprises restoration of all or a portion of a tissue or organ. The invention provides methods of promoting or enhancing regeneration. In some embodiments, the method of promoting or enhancing regeneration includes modulating one or more of proliferation, differentiation, dedifferentiation, innervation, survival, or migration. As used herein, the term “tissue regeneration” is used to generically refer to regeneration of one or more tissue types, including regeneration of complex multi-tissue units such as organs and appendages.

As used herein, the term “non-regenerative state” refers to tissues comprising cells that are terminally differentiated or otherwise refractory to renewal following injury or tissue. In some instances, a non-regenerative state refers to a naturally non-regenerating state in a non-regenerating organism or a refractory period in a regenerating organism. Non-regenerative cells, tissue, or organs may be derived from organisms, such as mammals, whose endogenous regenerative capacity is not robust. Alternatively, non-regenerative cells, tissue, or organs may be derived from endogenously non-regenerating cells or tissues derived from particular regions of otherwise robustly regenerative organisms. By way of example, a non-regenerative state includes a non-regenerative wound epithelium or scar tissue.

As used herein, the term “population of cells” refers to one or more cells in a tissue, organ, or culture. A population of cells may be manipulated or examined in vivo or in vitro.

The term “progenitor cell” is used synonymously with “stem cell”. Both terms refer to an undifferentiated cell which is capable of proliferation and giving rise to more progenitor cells having the ability to generate a large number of mother cells that can in turn give rise to differentiated, or differentiable daughter cells. In a preferred embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues.

The term “embryonic stem cell” is used to refer to the pluripotent stem cells of, for example, the inner cell mass of the mammalian embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

The term “adult stem cell” is used to refer to any multipotent stem cell derived from tissues other than the embryonic blastocyst. Adult stem cells include cells derived from non-blastocyst tissue, including tadpole, fetal, juvenile, and adult tissue. Stem cells have been isolated from a wide variety of adult tissues (e.g., non-blastocyst) including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the invention contemplates the identification of progenitor cells resident in any tissue, in any organism, during any stage of development. As used herein, the term “effective amount” means the total amount of the active component(s) of a composition or compound that is sufficient to cause a statistically significant change on a detectable biochemical or phenotypic characteristic. When applied to an individual active ingredient, administered alone, the term refers to that ingredient alone. When applied to a combination, the term refers to combined amounts of the active ingredients that result in the effect, whether administered in combination, serially or simultaneously.

The term “membrane” refers to phospholipid bilayers, which includes but is not limited to the cell membrane as well as other membranes of intracellular organelles, e.g., nucleus, golgi, mitochondria, etc.

The term “ion flux” refers to the movement of ions through an area/unit time. The term does not imply anything about the mechanism of ion movement. The term includes ion flux mediated by any ion transporter protein regardless of whether the transporter protein actively or passively shuttles ions. The term ion flux includes movement of ions into a cell or movement of ions out of a cells (e.g., efflux or influx). The term also includes ion flux mediated by an ionophore, in which an ion transporter protein is not required. Ionophores as described herein include chemical ionophores (mobile ion carriers) or channel formers. Some examples of Na⁺ ionophores include monensin and Gramicidin A.

As used herein, the terms “ion transporter proteins,” “transporter proteins,” “protein pumps” and “channel proteins” are used interchangeably and include proteins that mediate ion flux regardless of the particular ion species transported or the particular mechanism of action. The term includes proteins that are passive transporters, as well as proteins that are active transporters. “Class of ion transporter proteins” refers to categories of transporter proteins organized based on similar functional characteristics. For examples, a class of ion transporter proteins may include transporter proteins that transport a particular ion species (e.g., Ca, Cl, Na, H) or transporter proteins that transport a particular ion species using a particular mechanism of action.

### III. Detailed Description of Illustrative Embodiments

One of the goals of studying regeneration has been to understand the principles and processes that modulate regeneration in species capable of mounting robust regenerative responses, so that these principles and processes can be applied to increase the regenerative capacity of other organisms. The present invention contemplates the following novel concepts and methods by which regeneration may be achieved.

A. Modulation of Intracellular Sodium Concentration to Promote Cell Proliferation and/or Tissue Regeneration

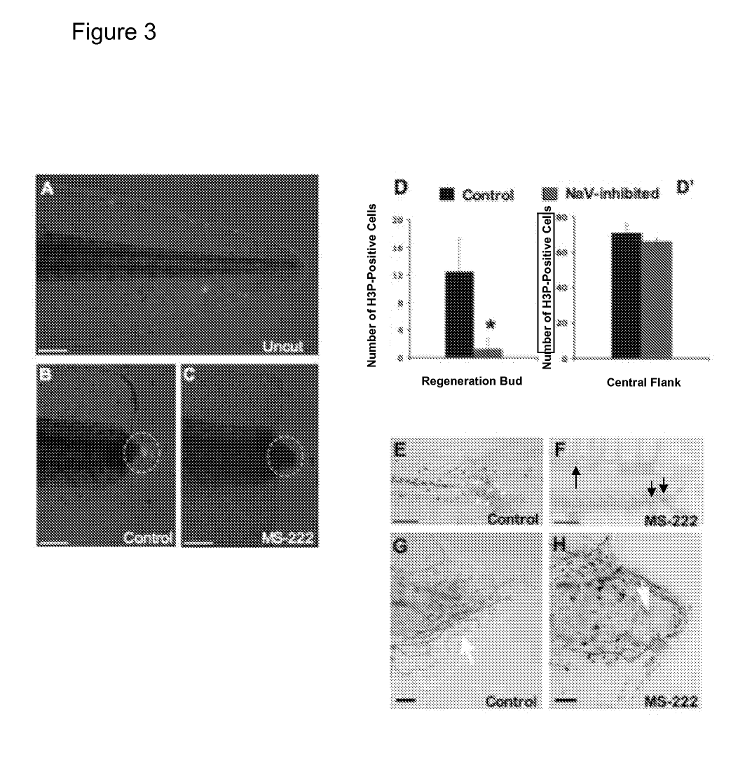

As demonstrated herein, inhibition of Na_{V}1.2 leads to, among other things, a failure to induce proliferation in the bud following amputation (FIG. 3D-F), and a reduction of innervation and a mispatterning of axonal growth (FIG. 3G-H). Thus, at least in the Xenopus tail, Na_{V}1.2 is the endogenously expressed voltage-gated sodium channel mediating sodium ion flux. However, the experiments detailed herein also revealed that intracellular sodium per se, and not Na_{V}1.2 expression or activity (or the expression or activity of any channel) modulates cell proliferation and regeneration.

Accordingly, one aspect of the invention provides a method of promoting one or more of proliferation or differentiation, comprising administering an amount of an agent effective to increase intracellular sodium concentration in a cell, wherein said agent induces Na⁺ influx into said cell, thereby promoting one or more of proliferation or differentiation, for example, of said cell. In certain embodiments, the cell is a stem cell, such as an embryonic stem cell or an adult stem cell.

In another aspect, the invention also provides a method of promoting tissue regeneration, comprising administering an amount of an agent effective to increase intracellular sodium concentration in a cell in a tissue, wherein said agent induces Na⁺ influx into said cell, thereby promoting cell proliferation to promote tissue regeneration. In certain embodiments, the method promotes innervation and/or vascularization of the tissue.

The intracellular sodium concentration may be influenced through one or more of the methods as described herein. In one example, an agent that opens a voltage-gated sodium channel (e.g., a voltage-gated sodium channel opener) may be used to promote influx of sodium. Such agents are well known in the art and are also disclosed in US Application No. 2008/0131920, incorporated herein by reference. In certain embodiments, the voltage-gated sodium channel is a Na_{V}1.2 channel, a Na_{V}1.5 channel, or an ENaC channel. However, any known voltage-gated sodium channel may be used. In some embodiments, the voltage-gated sodium channel is an endogenously expressed channel. Alternatively, the source of the voltage-gated sodium channel may be exogenously expressed (e.g., via transfection or electroporation). In a related embodiment, the exogenously expressed voltage-gated sodium channel may be a variant or fragment of the wild-type to confer different activity as compared to the wild-type version. When a voltage-gated channel is endogenously or exogenously expressed, the invention contemplates increasing intracellular sodium concentration by inducing sodium influx through the expressed voltage-gated sodium channel.

In other embodiments, the intracellular sodium concentration may also be influenced through the use of sodium ionophores or other agents that promote an increase in intracellular sodium concentration in a channel-independent manner. Ionophores as described herein include chemical ionophores (mobile ion carriers) or channel formers. Some examples of Na⁺ ionophores include monensin and Gramicidin A. In certain embodiments, insulin is used to influence the intracellular sodium concentration. The invention contemplates the use of agents that increase intracellular sodium concentration in a cell in a voltage-gated sodium channel-dependent or -independent manner.

In addition to the use of agents to alter intracellular sodium concentration, the method may further comprise administering the agent in the presence of a medium having a higher sodium concentration relative to the intracellular sodium concentration in the cell (or in the cell of a tissue or organ sample) prior to administration of one or more agents.

In certain embodiments, the cell (or cell of a tissue or organ sample) is in a non-regenerative state prior to administration of the agent. In other embodiments, the cell is a progenitor cell selected from one or more of an embryonic stem cell, a neural progenitor cell, a neural crest progenitor cell, a mesenchymal stem cell, or a muscle progenitor cell. In certain in vitro contexts, the method may further comprise administering one or more agents to a culture comprising the progenitor cell. Thus, the present invention provides, in certain embodiments, methods for promoting proliferation and/or differentiation of stem cells.

In some embodiments, the foregoing methods can be applied to regenerate all or a portion of a tissue (e.g., muscle or neuronal tissue), or an organ (e.g., kidney or pancreas), or structural unit (e.g., an appendage or spinal cord).

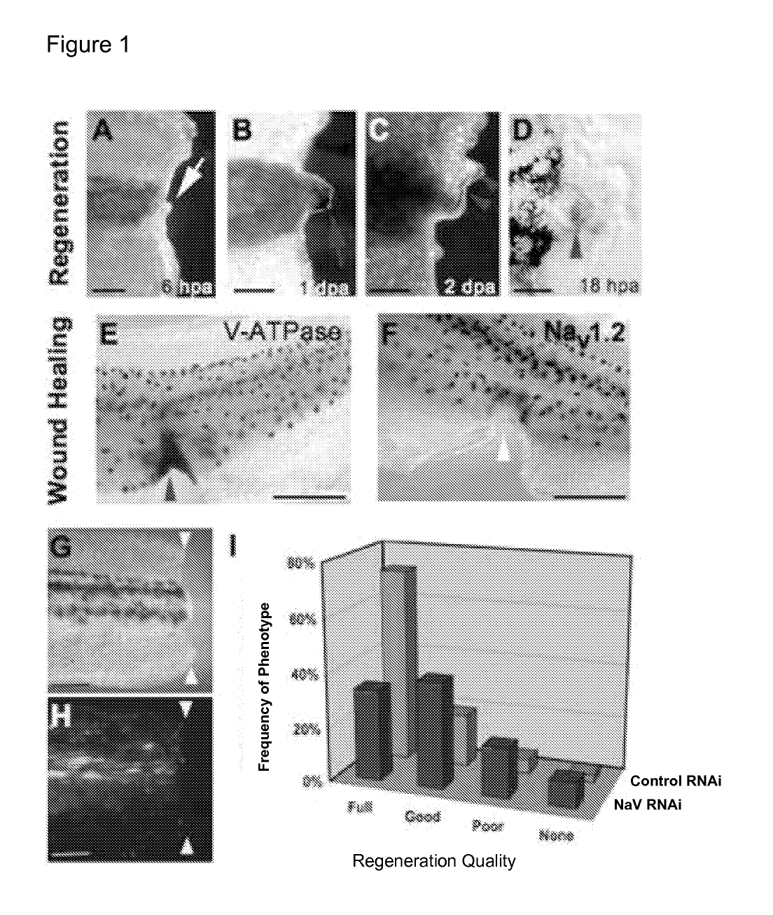

Further, as exemplified herein, genetic knockdown or pharmacological inhibition of Na_{V}1.2 both strongly abrogate regenerative ability in the absence of paralysis, general malformations or toxicity, or effects on primary tail development. Thus, as with a number of other transporters (Levin, 2006, 2007), it is possible to dissociate the housekeeping functions of this ion channel from subtle patterning roles. Notably, Na_{V}1.2 expression serves as a molecular marker distinguishing true regeneration from wound healing (FIG. 1D-F)—a distinction whose mechanistic details are often debated in the field of regeneration biology. Thus, in addition to the utility of the aspects and embodiments described herein, for example, to modulate cell proliferation, to modulate tissue regeneration, and as tools to study the biology of regeneration, an additional use of this technology is as a marker to identify and distinguish regeneration from wound healing. The development of such a marker is not only useful in the basic research concept, but also as the basis of a diagnostic tool that could be used to select appropriate treatments based on the state of an injury and the underlying tissue.

Accordingly, in another aspect, the invention provides a method for determining whether cells in a sample are in a wound healing state or in a regenerative state, comprising contacting said sample with a compound that detects expression of a Na_{V}1.2 channel, wherein a sample in which cells express the Na_{V}1.2 channel are identified as being in a regenerative state. By way of example, the sample may comprise the blastema of an amputated appendage, or any organ for which regeneration is desired. In some embodiments, the present method serves as a diagnostic tool to determine the fate of a given in vitro sample of cells or tissue to establish a therapeutic course. For example, using the present method, if it is determined that a tissue sample is undergoing true regeneration (e.g., of an organ or an appendage) and not simple wound healing, it would be a good candidate for transplant into an organism.

In a related embodiment, a compound that detects the Na_{V}1.2 channel may be one that detects the Na_{V}1.2 protein or Na_{V}1.2 mRNA. Some examples of such compounds include, for example, antibodies or nucleic acid probes that bind to the Na_{V}1.2 protein or hybridize to a nucleic acid encoding the Na_{V}1.2 protein, respectively. Reagents and methods for detection of protein or mRNA in a sample are well known in the art, and are further exemplified herein.

B. Modulation of Intracellular Sodium Concentration to Promote Cell Proliferation and/or Tissue Regeneration in a Non-Regenerative State

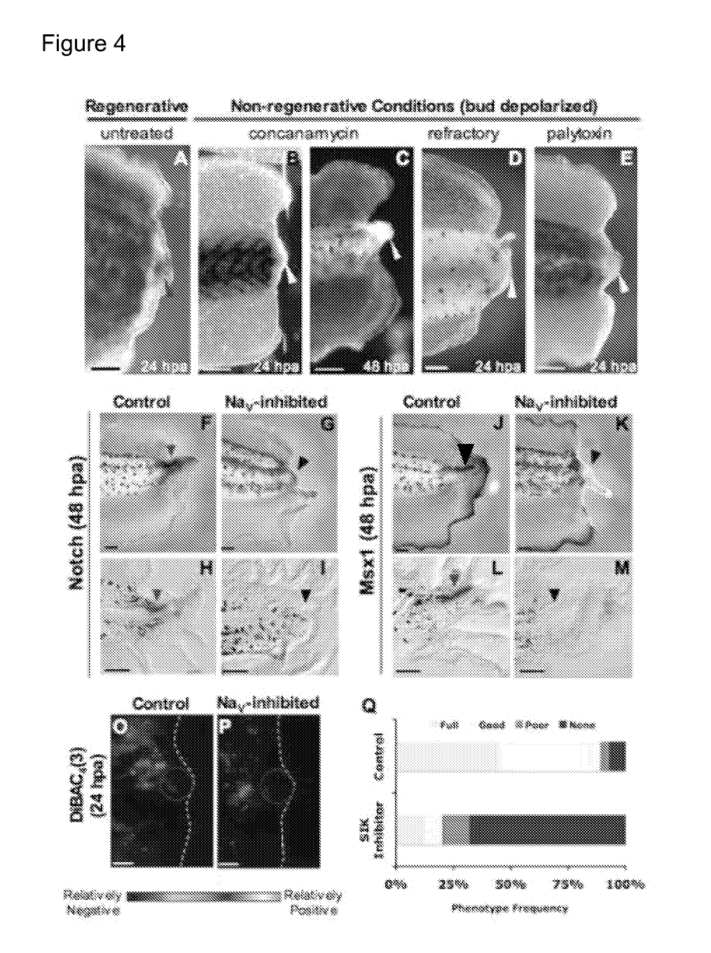

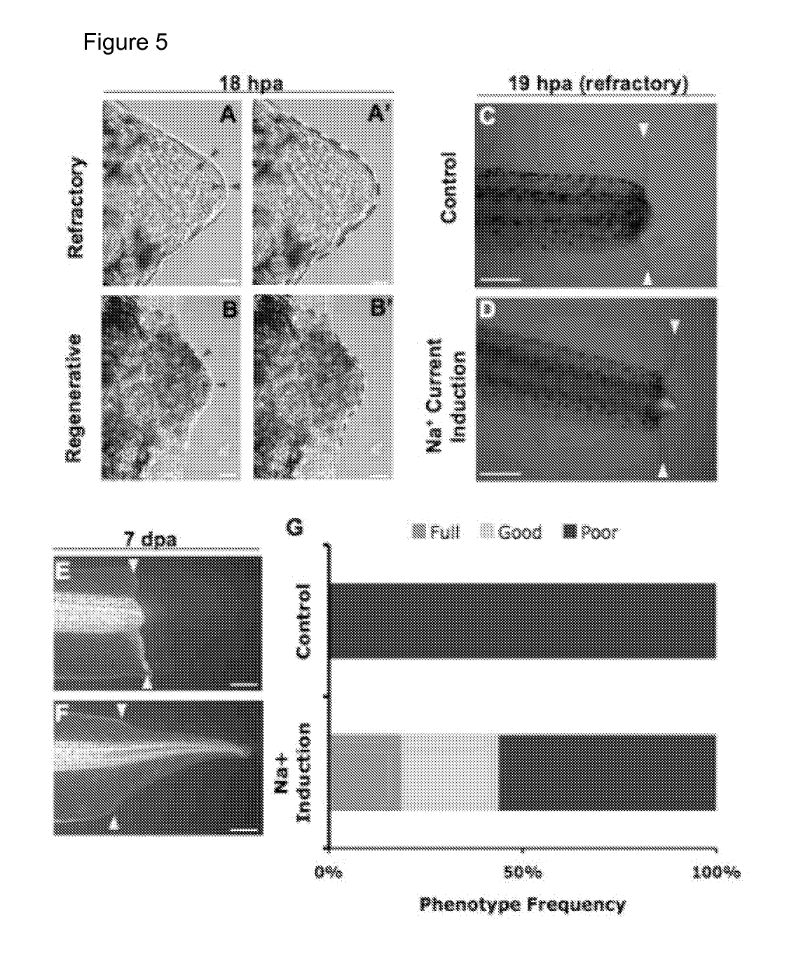

The Xenopus tadpole's refractory period provides a convenient context within which to test treatments that may overcome non-regenerative conditions in vertebrates. As shown herein, Na_{V}1.2 is not expressed in the bud during the refractory period (FIG. 4D). However, we showed that this could be overcome (FIG. 5) by a 1-hour pharmacological treatment that mimicked the transient NaV-dependent influx of sodium in bud cells and restored regeneration. The ability to restore regeneration using a rapid, highly controllable approach not requiring gene therapy with heterologous transporters or endogenous expression of such transporters is particularly valuable. Notable is the fact that induced regenerates rapidly formed normal tails of the correct size. The ability to induce a self-terminating and properly scaled growth program (producing neither tumor-like growth, nor an extra-large tail) suggests that sodium influx is a high-level target for therapeutic modulation because it is able to initiate complex, highly coordinated downstream morphogenetic programs in the host that do not require external micromanagement of the growth process. Also crucial is the fact that it was able to restore regeneration at 18 hpa during the refractory stage, long after a thick regeneration-incompetent wound epithelium has formed, suggesting that the cells of the non-regenerative bud remain competent to initiate regeneration even though they have been specified to not regenerate. This suggests that scarring and other non-regenerative responses to injury can be overcome and do not represent permanent blocks to productive tissue regeneration. Thus, modulating regenerative signals by increasing the intracellular sodium concentration may promote regeneration, generally, as well as in many non-regenerative conditions.

Accordingly, in some aspects, the invention provides a method of promoting tissue regeneration of a tissue in a non-regenerative state, comprising administering an amount of an agent effective to increase intracellular sodium concentration in a cell in said tissue, wherein said agent induces Na⁺ influx into said cell, thereby promoting cell proliferation to promote tissue regeneration. In certain embodiments, the method promotes innervation and/or vascularization of the tissue.

In another aspect, the invention also provides a method of promoting one or more of proliferation or differentiation of a cell that is in a non-regenerative state, comprising administering an amount of an agent effective to increase intracellular sodium concentration in a cell, wherein said agent induces Na⁺ influx into said cell, thereby promoting one or more of proliferation and/or differentiation.

As used herein, it is understood that a cell in a non-regenerative state refers to a cell or a population of cells derived from organisms, such as mammals, whose endogenous regenerative capacity is not robust. Alternatively, non-regenerative cells may be derived from endogenously non-regenerating cells or cells derived from particular regions of otherwise robustly regenerative organisms. By way of example, a non-regenerative state includes a non-regenerative wound epithelium or scar tissue.

The intracellular sodium concentration may be influenced through one or more of the methods as described herein. In one example, an agent that opens a voltage-gated sodium channel (e.g., a voltage-gated sodium channel opener) may be used to promote influx of sodium. Such agents are well known in the art and are also disclosed in US Application No. 2008/0131920, incorporated herein by reference. In certain embodiments, the voltage-gated sodium channel is a Na_{V}1.2 channel, a Na_{V}1.5 channel, or an ENaC channel. However, any known voltage-gated sodium channel may be used. In some embodiments, the voltage-gated sodium channel is an endogenously expressed channel. Alternatively, the source of the voltage-gated sodium channel may be exogenously expressed (e.g., via transfection or electroporation). In a related embodiment, the exogenously expressed voltage-gated sodium channel may be a variant or fragment of the wild-type to confer different activity as compared to the wild-type version. When a voltage-gated channel is endogenously or exogenously expressed, the invention contemplates increasing intracellular sodium concentration by inducing sodium influx through the expressed voltage-gated sodium channel.

In other embodiments, the intracellular sodium concentration may also be influenced through the use of sodium ionophores or other agents that promote an increase in intracellular sodium concentration in an channel-independent manner. Ionophores as described herein include chemical ionophores (mobile ion carriers) or channel formers. Some examples of Na⁺ ionophores include monensin and Gramicidin A. In certain embodiments, insulin is used to influence the intracellular sodium concentration. The invention contemplates the use of agents that increase intracellular sodium concentration in a cell in a voltage-gated sodium-independent manner. In some embodiments, the use of one or more sodium ionophore may be combined with exogenous expression of a voltage-gated sodium channel.

In addition to the use of agents to alter intracellular sodium concentration, the method may further comprise administering the agent in the presence of a medium having a higher sodium concentration relative to the intracellular sodium concentration in the cell (or in the cell of a tissue or organ sample) prior to administration of one or more agents. In some embodiments, the foregoing methods can be applied to regenerate all or a portion of a tissue (e.g., muscle or neuronal tissue), or an organ (e.g., kidney or pancreas), or structural unit (e.g., an appendage or spinal cord).

C. Modulation of Intracellular Sodium Concentration to Upregulate Genes that Promote Regeneration

As shown herein, inhibition of Na_{V}1.2 results in an abrogation of regeneration-specific gene expression such as MSX1 and Notch (FIG. 4F-M). Thus, Na_{V}1.2 drives regenerative growth endogenously, in part, by its regulation of downstream signaling genes including Notch1 and Msx1. These gene products appear to be important for building appendages during regeneration in other systems such as the tadpole limb, and zebrafish fin and heart regeneration (Poss et al., 2000; Poss et al., 2002). Consistent with the conserved roles of ion flows in regulating global patterning and morphogenetic cues (Levin, 2009; Nuccitelli et al., 1986), it is highly likely that the early initiating mechanism of ion transporter signaling in regeneration can also be utilized in other species.

Accordingly, in certain aspects, the invention provides a method of upregulating expression in a cell of one or more genes that promote tissue regeneration, comprising administering an amount of an agent effective to increase intracellular sodium concentration in a cell, wherein said agent induces Na⁺ influx into said cell, thereby upregulating expression in said cell of one or more genes that promote tissue regeneration. In certain embodiments, the method promotes innervation of the tissue. In certain embodiments, the method promotes vascularization of the tissue.

In another aspect, the invention also provides a method of upregulating expression in a cell of one or more genes that promote proliferation or differentiation, comprising administering an amount of an agent effective to increase intracellular sodium concentration in a cell, wherein said agent induces Na⁺ influx into said cell, thereby upregulating expression in said cell of one or more genes that promote proliferation or differentiation. In certain embodiments, the cell is a stem cell, such as an embryonic stem cell or an adult stem cell.

In certain embodiments, the present method regulates the expression of downstream signaling genes including, but not limited to, Notch1 and Msx1. However, it is understood that the present method can trigger the expression of other genes, e.g., BMP2, BMP4, and Delta, known to play an important function in tissue or organ regeneration. Such genes are well known in the art. In another embodiment, the present method also includes the exogenous expression (e.g., by transfection or electroporation) of a downstream signaling gene known to play an important function in tissue or organ regeneration in combination with methods described herein to increase intracellular sodium flux.

The intracellular sodium concentration may be influenced through one or more of the methods as described herein. In one example, an agent that opens a voltage-gated sodium channel (e.g., a voltage-gated sodium channel opener) may be used to promote influx of sodium. Such agents are well known in the art and are also disclosed in US Application No. 2008/0131920, incorporated herein by reference. In certain embodiments, the voltage-gated sodium channel is a Na_{V}1.2 channel, a Na_{V}1.5 channel, or an ENaC channel. However, any known voltage-gated sodium channel may be used. In some embodiments, the voltage-gated sodium channel is an endogenously expressed channel. Alternatively, the source of the voltage-gated sodium channel may be exogenously expressed (e.g., via transfection or electroporation). In a related embodiment, the exogenously expressed voltage-gated sodium channel may be a variant or fragment of the wild-type to confer different activity as compared to the wild-type version. When a voltage-gated channel is endogenously or exogenously expressed, the invention contemplates increasing intracellular sodium concentration by inducing sodium influx through the expressed voltage-gated sodium channel.

In other embodiments, the intracellular sodium concentration may also be influenced through the use of sodium ionophores or other agents that promote an increase in intracellular sodium concentration in an channel-independent manner. Ionophores as described herein include chemical ionophores (mobile ion carriers) or channel formers. Some examples of Na⁺ ionophores include monensin and Gramicidin A. In certain embodiments, insulin is used to influence the intracellular sodium concentration. The invention contemplates the use of agents that increase intracellular sodium concentration in a cell in a voltage-gated sodium-independent manner.

In addition to the use of agents to alter intracellular sodium concentration, the method may further comprise administering the agent in the presence of a medium having a higher sodium concentration relative to the intracellular sodium concentration in the cell (or in the cell of a tissue or organ sample) prior to administration of one or more agents.

In certain embodiments, the cell (or cell of a tissue or organ sample) is in a non-regenerative state prior to administration of the agent. In other embodiments, the cell is a progenitor cell selected from one or more of an embryonic stem cell, a neural progenitor cell, a neural crest progenitor cell, a mesenchymal stem cell, or a muscle progenitor cell. In certain in vitro contexts, the method may further comprise administering one or more agents to a culture comprising the progenitor cell. Thus, the present invention provides, in certain embodiments, methods for promoting proliferation and/or differentiation of stem cells.

In some embodiments, the foregoing methods can be applied to regenerate all or a portion of a tissue (e.g., muscle or neuronal tissue), or an organ (e.g., kidney or pancreas), or structural unit (e.g., an appendage or spinal cord).

### D. Modulation of Intracellular Sodium to Inhibit Cell Proliferation and Migration

As exemplified above, certain aspects of the disclosure provide numerous methods for increasing the intracellular sodium concentration in a cell (or in cells in a tissue) to promote proliferation, including proliferation leading to regeneration and innervation. Such methods and reagents have tremendous importance for the study of regeneration, the understanding of the molecular distinctions between regeneration and wound healing, and the development of treatments and approaches to promote regeneration in mammals as a therapeutic intervention for tissue loss due to disease, age, or injury.

Other aspects of the disclosure provide methods for inhibiting proliferation and/or migration of hyperproliferative cells. Exemplary hyper-proliferative cells are transformed cells and cancerous cells, such as cells existing in a tumor. By providing methods and reagents for decreasing cell proliferation and/or migration of these hyper-proliferative cells, the present disclosure provides methods for studying tumor growth and metastasis. Additionally, the present invention provides methods and reagents for developing treatments for cancers by reducing their growth (e.g., inhibiting proliferation) and/or by decreasing their migratory behavior (e.g., inhibiting metastasis). Exemplary hyper-proliferative cells and tissues that can be treated include, but are not limited to, solid tumors (such as those associated with breast cancer, lung cancer, pancreatic cancer, kidney cancer, colon cancer, ovarian cancer, liver cancer, stomach cancer, testicular cancer, and the like), blood cancers (such as leukemias and lymphomas), diffuse form tumors (such as melanomas and many brain tumors).

In another aspect, the disclosure provides a method for inhibiting one or more of proliferation or migration of a hyper-proliferative cell. The method comprises administering an amount of an agent effective to decrease intracellular sodium concentration in said hyperproliferative cell. The agent decreases intracellular sodium concentration by, for example, inducing Na⁺ efflux from said cell or preventing further sodium influx into said cell, thereby inhibiting one or more of proliferation or migration of said hyper-proliferative cell. In some embodiments, the agent may be a small molecule (e.g., a voltage-gated sodium channel blocker, or an ionophore that promotes Na⁺ efflux), or a nucleic acid (e.g., antisense or RNAi against a voltage-gated sodium channel for which silencing is desired). In a related embodiment, the agent may also be a polypeptide (e.g., a dominant negative variant of a hyperactive voltage-gated sodium channel).

In a related aspect, the disclosure provides a method of inhibiting growth and/or metastasis of a tumor. The method comprises administering an amount of an agent effective to decrease intracellular sodium concentration in cells in said tumor. The agent decreases intracellular sodium concentration by, for example, inducing Na⁺ efflux from said cell or preventing further sodium influx into said cell, thereby inhibiting cell proliferation to inhibit growth and/or metastasis of the tumor.

In certain embodiments of the foregoing, the agent induces Na+ efflux from said cell via an endogenously expressed voltage-gated sodium channel. In certain embodiments, the method further comprises administering said agent in the presence of a medium having a lower sodium concentration (external to the cell) relative to the intracellular sodium concentration in the cell prior to administration of said agent. In certain embodiments, the agent closes or disrupts a voltage-gated sodium channel to prevent further sodium influx into said cells.

In certain embodiments of any of the foregoing or following, the decrease in intracellular sodium concentration does not alter the membrane potential of said cell. In certain embodiments, the cell in which a decrease in intracellular sodium concentration is promoted does not express an exogenously introduced nucleic acid encoding a sodium channel. In certain embodiments, the cell in which a decrease in intracellular sodium concentration is promoted does not endogenously express a voltage-gated sodium channel. In certain embodiments, the cell in which a decrease in intracellular sodium concentration is promoted does endogenously express a voltage-gated sodium channel, and the method comprises administering an agent that disrupts the channel by inhibiting expression and/or activity of the channel.

In certain embodiments of any of the foregoing, the method inhibits growth of a tumor comprising cells in which the intracellular sodium concentration is modulated. In certain embodiments, the method inhibits migration and metastasis of a tumor comprising said cells.

In certain embodiments of any of the foregoing, the cells in which sodium concentration is modulated are tumor stem cells.

In certain embodiments of any of the foregoing, the method comprises administering said agent to a culture comprising said cell.

In certain embodiments of any of the foregoing, the agent is a small molecule.

When any of the foregoing is used as part of a method to study cancerous cells or tissue in vitro or to test such methods in vivo in human or animal models, the invention contemplates using the methods and reagents described herein alone or as part of a therapeutic regimen where one or more other drugs or treatment modalities are used.

The invention contemplates combinations of any of the foregoing aspects and embodiments of the invention.

### IV. Compounds That Modulate ion Flux

For any of the foregoing methods, the invention contemplates the use of any of a wide range of compounds to open or close a particular channel protein of interest to alter the ion flux of cells. Compounds that modulate the activity of particular ion transporters or classes of ion transporters are disclosed in US Application No. 2008/0131920, incorporated herein by reference. Such compounds may be used to modulate various ion channels according to the methods of the present invention.

In particular, the application contemplates the use of voltage-gated sodium channel openers to promote Na⁺ influx. The invention also discloses the use of any of a wide range of ionophores to alter the ion flux of cells. In particular, as demonstrated herein, monensin can be effectively used to promote influx of sodium. Other sodium ionophores known in the art, e.g., Gramicidin A, can also be used. Additionally, as demonstrated herein, insulin can also be used to promote influx of sodium.

Exemplary classes of compounds include, but are not limited to, small organic molecules, small inorganic molecules, nucleic acids (e.g., antisense oligonucleotides, RNAi constructs, ribozymes), peptides, polypeptides, peptidomimetics, and antibodies. For example, small compounds or antibodies may bind specifically to a particular channel protein, thereby activating or inactivating ion transport through that channel.

In other aspects, the application also contemplates the use of agents that inhibit, block, or otherwise disrupt voltage-gated sodium channels, including hyperactive voltage-gated sodium channels. In addition to small molecule blockers, as described below, various nucleic acids known in the art (e.g., antisense or RNAi) may also be used to disrupt or silence those voltage-gated sodium channels that are hyperactive or constitutively open, thereby causing hyperproliferative conditions (e.g., tumors). Further still, the invention also contemplates the overexpression of polypeptides that act as dominant negative forms of voltage-gated sodium channels, including hyperactive voltage-gated sodium channel. Methods of expressing desired polypeptides in cells are well known in the art.

Regardless of the specific agent used, in certain embodiments, one or more agents are used to alter ion flux, in particular, Na⁺ flux, through an endogenously or exogenously expressed ion channel, via a sodium ionophore (e.g., in a channel independent manner), or by other mechanisms (e.g., use of insulin). The result is an increase or decrease in intracellular sodium concentration.

### A. Antisense, Ribozyme and Triplex Techniques

Nucleic acid-based compounds include, but are not limited to, antisense oligonucleotides and ribozymes. Antisense oligonucleotides and ribozymes inhibit the expression of a protein, e.g., by inhibiting transcription and/or translation. In certain embodiments, the present invention contemplates the use of antisense compounds to disrupt the expression of a voltage-gated sodium channels, including a hyperactive voltage-gated sodium channel, thereby inhibiting hyper-proliferating cells such as those that occur in tumors.

Binding of the oligonucleotide or ribozyme to the nucleic acid encoding the particular channel protein for which inactivation is desired may be by conventional base pair complementarity, or, for example, in the case of binding to DNA duplexes, through specific interactions in the major groove of the double helix. In general, “antisense” therapy refers to the range of techniques generally employed in the art, and includes any therapy that relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a particular protein. Alternatively, the antisense construct is an oligonucleotide probe that is generated ex vivo and which, when introduced into the cell causes inhibition of expression by hybridizing with the mRNA and/or genomic sequences encoding a particular channel protein. Such oligonucleotide probes are preferably modified oligonucleotides that are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775).

The antisense oligonucleotide may comprise at least one modified base moiety known in the art. The antisense oligonucleotide may also comprise at least one modified sugar moiety selected from the group including, but not limited to, arabinose, 2-fluoroarabinose, xylulose, and hexose. The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. In yet a further embodiment, the antisense oligonucleotide is an -anomeric oligonucleotide. An—anomeric oligonucleotide forms specific double-stranded hybrids with complementary RNA in which, contrary to the usual-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides to be used in the present invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451).

The antisense molecules can be delivered to cells or animals in vitro or in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically. These and other methods are have been used to deliver single antisense oligonucleotides, as well as libraries of oligonucleotides.

Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

Ribozyme molecules designed to catalytically cleave an mRNA transcript can also be used to prevent translation of mRNA (See, e.g., PCT International Publication WO90/11364, published Oct. 4, 1990; Sarver et al., 1990, Science 247:1222-1225 and U.S. Pat. No. 5,093,246), and is well known in the art. As in the antisense approach, the ribozymes can be composed of modified oligonucleotides (e.g., for improved stability, targeting, etc.) and can be delivered in vivo or in vitro. Alternatively, endogenous gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Antisense RNA and DNA, ribozyme, and triple helix molecules to be used in the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

### B. RNAi

In other embodiments, the compound is an RNAi construct. The present invention contemplates the use of RNAi compounds to disrupt the expression of a voltage-gated sodium channels, including a hyperactive voltage-gated sodium channel, thereby inhibiting hyper-proliferating cells such as those that occur in tumors. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties, as specifically exemplified herein.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

Production of RNAi constructs to be used in the present invention can be carried out by chemical synthetic methods or by recombinant nucleic acid techniques known in the art. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

The siRNA molecules to be used in the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

Exemplary RNAi constructs that specifically recognize a particular gene, or a particular family of genes can be selected using methodology outlined in detail above with respect to the selection of antisense oligonucleotide. Similarly, methods of delivering RNAi constructs include the methods for delivering antisense oligonucleotides outlined in detail above, and as exemplified herein.

### V. Cells and Animals

As outlined throughout in reference to particular methods of the invention, the subject methods can be conducted in vitro, in vivo, or ex vivo in cells, tissue, or organs derived from or resident in virtually any organism.

The foregoing methods can be conducted in cells in culture, in tissue samples maintained ex vivo, or in animals. When the method is conducted using cells in culture, the invention contemplates using cells derived from any organism, tissue, or stage of development. Furthermore, the invention contemplates that the cells may be primary cultures of cells, or transformed cell lines, and that the cells can either be wild type cells or cells containing one or more mutations. Mutant cells or cell lines may be models of a particular disease or injury, or may be derived from animals having a specific disease or injury (e.g., cancer cells harvested from an animal).

Cells may be derived from (e.g., derived from and cultured in vitro as populations of cells or tissues) or reside in (cells resident in a whole animal or portion of a whole animal) any of a number of animal species. Exemplary animals include, but are not limited to, flatworms, amphibians, fish, reptiles, birds, or mammals. Suitable flatworms include planarian. Suitable amphibians include Xenopus laevis, Xenopus tropicalis, and other species of frog. Suitable birds include chickens, as well as other birds commonly used or maintained in a laboratory setting. Suitable mammals include mice, rats, hamsters, goats, sheep, pigs, cows, dogs, cats, rabbits, non-human primates, and humans.

Regardless of the species of cells or animal selected, the invention contemplates that cells may be derived from or reside in an animal of virtually any stage of development. For example, the cells may be derived from or reside in an embryonic, larval, fetal, juvenile, or adult organism.

To further illustrate, in one embodiment, the foregoing methods may be conducted using cells derived from or resident in a nematode. There are over 10,000 known nematode species. These include parasitic nematodes (e.g., nematodes that are parasitic to humans, non-human animals, or plants). Exemplary parasitic nematodes include, but are not limited to, whipworms, Ascaris, hookworms, filarial worms, and root knot nematodes. C. elegans is perhaps the most well known and thoroughly studied nematode, and the invention contemplates using C. elegans or other nematodes.

In another embodiment, the foregoing methods may be conducted in cells derived from or resident in a fish or amphibian species. Zebrafish (e.g., adult zebrafish and developing, e.g., embryonic fish) are a particular example of a fish well suited for study. Zebrafish are an extensively used developmental system, and genetic, cell biological, and molecular biological reagents and methods are well known and available. Additionally, numerous chemical and radiation-based screens have produced large numbers of mutant zebrafish that can also be used for study.

Xenopus laevis and Xenopus tropicalis (e.g., adult, embryonic, tadpole, etc. stage animals) are particular examples of amphibians well suited for study. Both species are used extensively, and well developed reagents exist. For example, the availability of these molecular reagents facilitates assays based on changes in gene or protein expression, either instead of or in addition to assays based on morphological criteria. Additionally, Xenopus tropicalis is a genetically tractable model organism, and mutants have been and continue to be generated and characterized. Xenopus cells and whole organisms are excellent systems for screening assays. The cells of early Xenopus embryos are relatively large, and thus easily manipulated, injected, and used for electrophysiological recording. Eggs and embryos can be collected in very large numbers. This facilitates biochemical, pharmacological, and statistical analyses.

In another embodiment, the foregoing methods may be conducted in cells derived from or resident in a flatworm. Exemplary flatworms are the free-living (e.g., non-parasitic) flatworm planaria. Planaria are in the phylum Platylhelmenthes and the class Turbellaria. There are numerous species of planaria, any of which can be readily used. Planaria exhibit much of the complexity of vertebrate systems: a well-differentiated nervous system, intestine, eyes, brain, three tissue layers, and bilateral symmetry. Planaria represent a critical breakthrough in the evolution of the animal body plan and are thought to very closely resemble the proto-bilaterian ancestor. It is the first organism to have both bilateral symmetry and encephalization, making it capable of detecting environmental stimuli quicker and more efficiently than the lower metazoans. Despite a simplistic appearance and evolutionary position, planaria possess a well-developed nervous system with true synaptic transmission and have what can be considered the first animal “brain” (Sarnat and Netsky (1985) Can J Neurol Sci. 12(4): 296-302). They have also developed sensory capabilities for the detection of light (Brown and Park (1975) Int J. Chronobiol. 3(1):57-62; Brown et al., 1968), chemical gradients (Mason (1975) Anim Behav. May; 23(2): 460-9; Miyamoto and Shimozawa (1985) Zoological Science (Tokyo) 2: 389-396), vibration (Fulgheri and Messeri (1973) Boll Soc Ital Biol Sper. 49(20): 1141-5), electric fields (Brown and Ogden (1968) J Gen Physiol. 51(2)255-60), magnetic fields (Brown and Chow (1975) Physiological Zoology 48: 168-176; Brown (1966) Nature 209: 533-5), and weak γ-radiation (Brown and Park (1964) Nature 202: 469-471).

Planaria have exceptional regenerative capacity. A bisected flatworm readily regenerates. Thus, planaria, either whole animals or fragments, serve as an excellent model system in which to study the implications of ion flux on cell proliferation, differentiation, innervation, and migration. In addition to planaria, other model systems have enhanced regenerative capacity, and these systems are especially well suited for studies of ion flux on regeneration. By way of example, fish and amphibian species may be especially useful as model systems in such studies.

The invention contemplates the use of animals, including any of the foregoing animals and cells derived therefrom.

Regardless of the particular organism selected, and regardless of whether the subject methods are conducted in vitro or in vivo, cells, tissue, organs, or animals may be of any developmental stage including, but not limited to, embryonic, fetal, larval, tadpole, juvenile, and adult stage cells or organisms. One of skill in the art can select the proper animal and developmental stage depending on the application of the subject method. Furthermore, one of skill in the art can select the appropriate animal and developmental stage based on the research interests of the investigator, time, and cost considerations, as well as the availability of other complementary research reagents. Additionally, even when whole organisms or large fragments of whole organisms are used, one of skill in the art may choose to examine a particular biological process in only a portion of the whole organism or fragment.

In one embodiment, the animal, tissue, or organ (including whole animals, injured animal, fragments, or cell derived therefrom) is selected based on its robust regenerative ability. Cells, tissues, organs, or animals with an enhanced regenerative ability may be useful in methods for identifying and characterizing a role for ion transporter proteins, ion flux, membrane potential, and/or pH in dedifferentiation and regeneration. Exemplary animals and systems with enhanced regenerative capacity include, but are not limited to, planaria, the zebrafish tail, the amphibian (e.g., Xenopus) tail, and the amphibian limb. An understanding of how regeneration is modulated in any of these systems can be used to increase/stimulate regenerative capacity in organisms and systems whose endogenous regenerative capacity is less robust. In another embodiment, the animal or tissue is selected for screening and study specifically because its endogenous regenerative capacity is not robust. Such systems include any cells, tissues, or organs derived from organisms, such as mammals, whose endogenous regenerative capacity is not robust. Such systems also include endogenously non-regenerating cells or tissues derived from particular regions of otherwise robustly regenerative organisms.

In one embodiment, the animal/organism is a protochordate. Protochordates possess a hollow dorsal nerve cord, gill slits, and a notochord. Exemplary protochordates include tunicata (e.g., sea squirts, etc.) and cephalochordate (e.g., amphioxus). Exemplary amphioxus include, but are not limited to Ciona intestinalis and Branchiostoma floridae (Holland and Gibson-Brown (2003) BioEssays 25: 528-532; Gostling and Shimeld (2003) Evolution and Development 5: 136; Dehal et al. (2002) Science 298: 2157-2167; Nishiyama et al. (1972) Tohoku J Exp Med 107: 95-96; Ogasawara et al. (2002) Develop Genes Evol 212: 173-185; Pope and Rowley (2002) J Exp Biology 205: 1577-1583).

In another embodiment, the animal/organism is a hemichordate. Exemplary hemichordates include acorn worms (Tagawa et al. (2001) Evol and Develop 3: 443).

In another embodiment, the animal/organism is a nematode. There are over 10,000 known nematode species. These include parasitic nematodes (e.g., nematodes that are parasitic to humans, non-human animals, or plants). Exemplary parasitic nematodes include, but are not limited to, whipworms, Ascaris, hookworms, filarial worms, and root knot nematodes.

C. elegans is perhaps the most well known and thoroughly studied nematode, and the invention contemplates using C. elegans or other nematodes. Although C. elegans is considered a soil nematode, methods for culturing C. elegans in various quantities of liquid media (e.g., in a fluid) are well developed. See, http://elegans.swmed.edu/. Accordingly, the methods and apparatuses of the invention for conducting assays in aquatic animals can be readily used to conduct assays in C. elegans.

In another embodiment, the animal is a fish or amphibian. Exemplary amphibians include frog (e.g., species of Xenopus) and salamanders (e.g., species of Axolotls).

In another embodiment, the animal is a flatworm. Exemplary flatworms are the free-living (e.g., non-parasitic) flatworm planaria. Planaria are in the phylum Platylhelmenthes and the class Turbellaria. There are numerous species of planaria, any of which can be readily used.

In another embodiment, the organism is a mammal such as a mouse, rat, rabbit, pig, cow, dog, cat, non-human primate, or human.

The invention contemplates the use of any of the foregoing animals. Each of these has numerous characteristics that make them suitable for particular assays or for particular methods of promoting/inhibiting proliferation, differentiation, and/or migration. The appropriate model organism can be readily selected based on the particular assays being conducted, as well as space and resource constraints. Furthermore, the appropriate developmental stage can be readily selected. Exemplary developmental stages include, but are not limited to, embryonic stages, fetal stages, tadpole stages, larval stages, juvenile stages, and adult stages. In certain embodiments, the animal is chosen due to its optical accessibility. Furthermore, the invention contemplates studying whole animals, animal fragments, or animals inflicted with an injury. An exemplary animal fragment is a bisected or trisected organism. In one embodiment, the animal fragment is a bisected or trisected planarian. Additionally, the invention contemplates the use of wild type or mutant animals. In one embodiment, the animal is a wild type embryonic, tadpole, larval, fetal, juvenile, or adult stage animal. In another embodiment, the animal is a mutant embryonic, tadpole, larval, fetal, juvenile, or adult stage animal.

In certain embodiments, it may be desirable to conduct an assay, for example an assay to identify and/or characterize a compound that modulates a particular developmental or regenerative process, in a relatively simple system. Identified compounds or candidate ion transporter proteins can later be analyzed in higher organisms including mice, rats, non-human primates, and humans.

In certain other embodiments, it may be desirable to conduct an assay in parallel using different populations of cells. For example, screening assays can be conducted in parallel using cells derived from or resident in different organisms. Alternatively, screening assays can be conducted in parallel using cells of varying developmental stages derived from or resident in the same organism. In still another embodiment, screening assays can be conducted in parallel using cells of different developmental lineages (e.g., different cell or tissue types) derived from or resident in the same model organism. In this embodiment, the cells of differing developmental lineages can be of the same or varying developmental stages.

In certain embodiments, the cells are stem cells. Exemplary stem cells include embryonic stem cells, adult stem cells, and tumor stem cells. Such stem cells can be from any tissue, organism or stage of development.

Depending on the particular model system and biological process chosen (e.g., organism, cell type, developmental stage, etc) for study or manipulation, one of skill in the art can select the appropriate culture conditions and methods for monitoring changes in the model system. For example, certain phenotype changes can be observed and monitored based on visual inspection with either the aided or unaided eye. Other phenotypic changes can be observed using molecular, cell biological, or biophysical reagents available in the art. For example, changes in the expression of one or more molecular markers can be assessed using known techniques including, but not limited to, RT-PCR, in situ hybridization, Northern blot analysis, Western blot analysis, immunocytochemistry, immunohistochemistry, and GeneChip analysis. Further tools including, but not limited to, method of detecting changes in cell proliferation, cell death, cell survival, membrane potential, intracellular pH, ion flux and the like can also be used to detect and assess phenotypic changes in cells or organisms.

### VI. Exemplary Disease and Injuries

Compounds, and pharmaceutical preparations thereof, that modulate intracellular sodium concentration in cells to induce proliferation, differentiation, innervation, and/or migration of cells may be useful in the treatment of injury requiring regeneration or degenerative disease. Such compounds can be administered to a human or non-human patient in need of augmenting a regenerative response to disease or injury. Briefly, compounds that promote regeneration may be administered to promote the combination of proliferation, differentiation, innervation, and/or migration of cells needed to regenerate damaged, diseased, or injured tissue.

The invention contemplates the use of compounds individually or in combination. Suitable combinations include combinations of multiple compounds identified as promoting proliferation, differentiation, and/or migration of cells by modulating intracellular sodium concentration. Suitable combinations also include a compound that promotes proliferation, differentiation, and/or migration by modulating intracellular sodium concentration along with one or more agents conventionally used in the treatment of the particular injury or degenerative disease.

Multiple agents may act additively or synergistically, and include combinations of agents that may show little or no effect when administered alone. Furthermore, the invention contemplates the use of agents in combination with known factors that influence proliferation, differentiation, innervation, migration, or survival of a particular cell type. Still further, the invention contemplates the use of agents as part of a therapeutic regimen along with other surgical, radiological, chemical, homeopathic, or pharmacologic intervention appropriate for the particular cell type, disease or condition.

Agents which possess one of more of these characteristics may be useful in a therapeutic context. For example, injuries and diseases of the central and peripheral nervous system effect a tremendous number of people and exact a large financial and person toll. Injuries include traumatic injuries (i.e., breaks, blunt injury, burns, lacerations) to the brain or spinal cord, as well as other injuries to any region of the CNS or PNS including, but not limited to, injuries caused by bacterial infection, viral infection, cell damage following surgery, exposure to a toxic agent, cellular damage caused by cancer or other proliferative disorder, ischemia, hypoxia, and the like. Currently, effective treatments for injuries of the CNS and PNS are limited, and individuals often experience long-term deficits consistent with the extent of injury, the location of the injury, and the types of cell that are effected.

In addition to injuries of the CNS and PNS, there are a wide variety of neurodegenerative diseases that effect particular regions and/or cell types of the CNS or PNS. These diseases are often progressive in nature, and individuals afflicted with many of these diseases have few treatment options at there disposal. Exemplary neurodegenerative diseases include, but are not limited to, Parkinson's disease, Huntington's disease, Alzheimer's disease, ALS, multiple sclerosis, stroke, macular degeneration, peripheral neuropathy, and diabetic neuropathy.

In certain embodiments, compounds can be administered to promote regeneration of mesodermal or endodermal cell and tissue types. Injuries and diseases of tissues derived from the mesoderm or endoderm include, but are not limited to, myocardial infarction, osteoarthritis, rheumatoid arthritis, diabetes, cirrohsis, polycystic kidney disease, inflammatory bowel disease, pancreatitis, Crohn's disease, cancer of any mesodermal or endodermal tissue (e.g, pancreatic cancer, Wilms tumor, soft cell carcinoma, bone cancer, breast cancer, prostate cancer, ovarian cancer, uterine cancer, liver cancer, colon cancer, etc), and injuries to any mesodermal or endodermal tissue including breaks, tears, bruises, lacerations, burns, toxicity, bacterial infection, and viral infection.

Furthermore, agents identified by the methods of the present invention may be used to modulate cells of the blood and blood vessels. Exemplary agents can be used to modulate (promote or inhibit) angiogenesis. Inhibition of angiogenesis is of particular use in the treatment of many forms of cancers, as well as in conditions aggravated by excess angiogenesis such as macular degeneration. Promotion of angiogenesis is of particular use in the treatment of conditions caused or aggravated by decreased blood flow. Exemplary conditions include, but are not limited to, myocardial infarction, stroke, and ischemia. Additionally, agents identified by the methods of the present invention can be used to promote proliferation and differentiation of various cell types of the blood and can be used in the treatment of anemia, leukemia, and various immunodeficiencies.

For any of the foregoing, the application contemplates that agents may be administered alone, or may be administered in combination with other agents. Further, the application contemplates that agents identified according to the subject methods can be administered as part of a therapeutic regimen along with other treatments appropriate for the particular injury or disease being treated. For example, in the case of Parkinson's disease, a subject agent may be administered in combination with L-dopa or other Parkinson's disease medications, or in combination with a cell based neuronal transplantation therapy for Parkinson's disease. In the case of an injury to the brain or spinal cord, a subject agent may be administered in combination with physical therapy, hydrotherapy, massage therapy, and the like. In the case of peripheral neuropathy, as for example diabetic neuropathy, a subject agent may be administered in combination with insulin. In the case of myocardial infarction, the subject agent may be administered along with angioplasty, surgery, blood pressure medication, and/or as part of an exercise and diet regimen.

Physical injuries may result in cellular damage that ultimately limits the function of a particular cell or tissue. For example, physical injuries to cells in the CNS may limit the function of cells in the brain, spinal cord, or eye. Examples of physical injuries include, but are not limited to, crushing or severing of neuronal tissue, such as may occur following a fall, car accident, gun shot or stabbing wound, etc. Further examples of physical injuries include those caused by extremes in temperature such as burning, freezing, or exposure to rapid and large temperature shifts.

Physical injuries to mesodermal cell types include injuries to skeletal muscle, cardiac muscle, tendon, ligament, cartilage, bone, and the like. Examples of physical injuries include, but are not limited to, crushing, severing, breaking, bruising, and tearing of muscle tissue, bone or cartilage such as may occur following a fall, car accident, gun shot or stabbing wound, etc. Further examples of physical injuries include breaking, tearing, or bruising of muscle tissue, bone, cartilage, ligament, or tendon as may occur following a sports injury or due to aging. Further examples of physical injuries include those caused by extremes in temperature such as burning, freezing, or exposure to rapid and large temperature shifts.

Physical injuries to endodermal cell types include injuries to hepatocytes and pancreatic cell types. Examples of physical injuries include, but are not limited to, crushing, severing, and bruising, such as may occur following a fall, car accident, gun shot or stabbing wound, etc. Further examples of physical injuries include those caused by extremes in temperature such as burning, freezing, or exposure to rapid and large temperature shifts.

Further examples of an injury to any of the aforementioned cell types include those caused by infection such as by a bacterial or viral infection. Examples of bacterial or viral infections include, but are not limited to, meningitis, staph, HIV, hepatitis A, hepatitis B, hepatitis C, syphilis, human papilloma virus, strep, etc. However, one of skill in the art will recognize that many different types of bacteria or viruses may infect cells and cause injury.

The methods of the present invention may be applied to regenerate all or a portion of a tissue or organ. That is, in addition to the types of tissue (muscle tissue, neural tissue, skin, bone, cartilage, ligament, or tendon) that may be regenerated according to the present invention, the invention may be applied to regenerate all or a portion of an organ or a structure. Some examples of organs and structures include, but are not limited to, the following: liver, pancreas, kidney, heart, spinal cord, limbs, and digits.

Additionally, injury to a particular cell type may occur as a consequence or side effect of other treatments being used to relieve some condition in an individual. For example, cancer treatments (chemotherapy, radiation therapy, surgery) may cause significant damage to both cancerous and healthy cells. Surgery; implantation of intraluminal devices; the placement of implants, pacemakers, shunts; and the like can all result in cellular damage.

A wide range of neurodegenerative diseases cause extensive cell damage (i.e., injury) to cells of the CNS and PNS. Accordingly, neurodegenerative diseases are candidates for treatment using the described agents. Administration of a subject agent can promote neuronal regeneration in the CNS or PNS of a patient with a neurodegenerative disease, and the promotion of neuronal regeneration can ameliorate, at least in part, symptoms of the disease. Agents may be administered individually, in combination with other agents of the invention, or as part of a treatment regimen appropriate for the specific condition being treated. The following are illustrative examples of neurodegenerative conditions which can be treated using the subject agents.

Parkinson's disease is the result of the destruction of dopamine-producing neurons of the substantia nigra, and results in the degeneration of axons in the caudate nucleus and the putamen degenerate. Although therapies such as L-dopa exist to try to ameliorate the symptoms of Parkinson's disease, to date we are unaware of treatments which either prevent the degeneration of axons and/or increase neuronal regeneration. Administration of agents with promote neuronal regeneration can help to ameliorate at least certain symptoms of Parkinson's disease including rigidity, tremor, bradykinesia, poor balance and walking problems.

Alzheimer's disease, a debilitating disease characterized by amyloid plaques and neurofibrillary tangles, results in a loss of nerve cells in areas of the brain that are vital to memory and other mental abilities. There also are lower levels of chemicals in the brain that carry complex messages back and forth between nerve cells. Alzheimer's disease disrupts normal thinking and memory. The incidence of Alzheimer's disease will only increase as the average life expectancy continues to rise around the world. One of the most notable features of Alzheimer's disease is that affected individuals can live for extended periods of time (ten or more years) while being in an extremely debilitated state often requiring round the clock care. Accordingly, the disease takes not only an enormous emotional toll, but also exacts a tremendous financial toll on affected individuals and their families. Therapies which improve neuronal function have substantial utility in improving the quality of life of Alzheimer's sufferers.

Huntington's disease is a degenerative disease whose symptoms are caused by the loss of cells in a part of the brain called the basal ganglia. This cell damage affects cognitive ability (thinking, judgment, memory), movement, and emotional control. Symptoms appear gradually, usually in midlife, between the ages of 30 and 50. However, the disease can also strike young children and the elderly. Huntington's disease is a genetic disorder. Although people diagnosed with the disease can often maintain their independence for several years following diagnosis, the disease is degenerative and eventually fatal. Currently, there are no treatments available to either cure or to ameliorate the symptoms of this disease. Furthermore, the onset of Huntington's disease is typically in middle-age (approx age 40), at a time when many people have already had children. Thus, people have usually passed this fatal genetic disorder to their off-spring before they realize that they are ill.

Amyotrophic lateral sclerosis (ALS), often referred to as “Lou Gehrig's disease,” is a progressive neurodegenerative disease that attacks motor nerve cells in the brain and the spinal cord. Degeneration of motor neurons affect the ability of the brain to initiate and control muscle movement. With all voluntary muscle action affected, patients in the later stages of the disease become totally paralyzed, and eventually die.

Multiple sclerosis (MS) is an illness diagnosed in over 350,000 persons in the United States today. MS is characterized by the appearance of more than one (multiple) areas of inflammation and scarring of the myelin in the brain and spinal cord. Thus, a person with MS experiences varying degrees of neurological impairment depending on the location and extent of the scarring. The most common characteristics of MS include fatigue, weakness, spasticity, balance problems, bladder and bowel problems, numbness, vision loss, tremor and vertigo. The specific symptoms, as well as the severity of these symptoms, varies from patient to patient and is largely determined by the particular location within the brain of the lesions.

MS is considered an autoimmune disease. Recent data suggest that common viruses may play a role in the onset of MS. If so, MS may be caused by a persistent viral infection or alternatively, by an immune process initiated by a transient viral infection in the central nervous system or elsewhere in the body. Epidemiological studies indicating the distribution of MS patients suggest that there is a triggering factor responsible for initiating onset of the disease. Without being bound by theory, it appears that some environmental factor, most likely infectious, must be encountered.

The incidence of MS is higher in North America and Europe and this geographic distribution is further suggestive of an environmental influence(s) underlying onset of MS. Additionally, MS is more prevalent in women than in men, and is more common amongst Caucasians than within either Hispanic or African-American populations. Interestingly, MS is extremely rare within Asian populations.

Macular degeneration is a catch-all term for a number of different disorders that have a common end result: the light-sensing cells of the central region of the retina—the macula—malfunction and eventually die, with gradual decline and loss of central vision, while peripheral vision is retained. Most cases of macular degeneration are isolated, individual, occurrences, mostly in people over age 60. These types are called Age Related Macular Degeneration (AMD). More rarely however, younger people, including infants and young children, develop macular degeneration, and they do so in clusters within families. These types of macular degeneration are collectively called Juvenile Macular Degeneration and include Stargardt's disease, Best's vitelliform macular dystrophy, Doyne's honeycomb retinal dystrophy, Sorsby's fundus dystrophy, Malattia levintinese, Fundus flavimaculatus, and Autosomal dominant hemorrhagic macular dystrophy.

The present invention makes available effective therapeutic agents for restoring cartilage function to a connective tissue. Such methods are useful in, for example, the repair of defects or lesions in cartilage tissue which is the result of degenerative wear such as that which results in arthritis, as well as other mechanical derangements which may be caused by trauma to the tissue, such as a displacement of torn meniscus tissue, meniscectomy, a laxation of a joint by a torn ligament, misalignment of joints, bone fracture, or by hereditary disease. The present reparative method is also useful for remodeling cartilage matrix, such as in plastic or reconstructive surgery, as well as periodontal surgery. The present method may also be applied to improving a previous reparative procedure, for example, following surgical repair of a meniscus, ligament, or cartilage. Furthermore, it may prevent the onset or exacerbation of degenerative disease if applied early enough after trauma.

Such connective tissues as articular cartilage, interarticular cartilage (menisci), costal cartilage (connecting the true ribs and the sternum), ligaments, and tendons are particularly amenable to treatment. As used herein, regenerative therapies include treatment of degenerative states which have progressed to the point of which impairment of the tissue is obviously manifest, as well as preventive treatments of tissue where degeneration is in its earliest stages or imminent. The subject method can further be used to prevent the spread of mineralisation into fibrotic tissue by maintaining a constant production of new cartilage.

In an illustrative embodiment, the subject method can be used to treat cartilage of a diarthroidal joint, such as a knee, an ankle, an elbow, a hip, a wrist, a knuckle of either a finger or toe, or a temperomandibular joint. The treatment can be directed to the meniscus of the joint, to the articular cartilage of the joint, or both. To further illustrate, the subject method can be used to treat a degenerative disorder of a knee, such as which might be the result of traumatic injury (e.g., a sports injury or excessive wear) or osteoarthritis.

In still further embodiments, agents of the present invention can be employed for the generation of bone (osteogenesis) at a site in the animal where such skeletal tissue is deficient. For instance, administration of an agent that promotes the differentiation of stem cells to bone can be employed as part of a method for treating bone loss in a subject, e.g. to prevent and/or reverse osteoporosis and other osteopenic disorders, as well as to regulate bone growth and maturation. For example, preparations comprising the identified agents can be employed, for example, to induce endochondral ossification.

Therapeutic compositions can be supplemented, if required, with other osteoinductive factors, such as bone growth factors (e.g. TGF-β factors, such as the bone morphogenetic factors BMP-2 and BMP-4, as well as activin), and may also include, or be administered in combination with, an inhibitor of bone resorption such as estrogen, bisphosphonate, sodium fluoride, calcitonin, or tamoxifen, or related compounds.

In still further embodiments, the disclosure provides methods and reagents for inhibiting proliferation or migration of hyper-proliferative cells. Such methods and reagents can be used in the treatment of virtually any type of cancer. Exemplary cancers include, but are not limited to: solid tumors (such as tumors of the liver, kidney, lung, breast, stomach, colon, ovary, testes, bladder, or pancreas), blood cancers (such as leukemias and lymphomas), and diffuse tumors (such as melanomas and certain brain tumors). The methods and reagents described herein can be used alone or in combination with one or more additional cancer treatments appropriate for the particular hyper-proliferative disorder. Exemplary other treatments include, but are not limited to, chemotherapeutic agents, immunomodulating agents, radiation therapy, surgery, acupuncture, massage, hormone therapy, pain management, exercise, and dietary therapy.

### VII. Models of Regeneration

Proliferation and regeneration can be studied in vitro using primary or transformed cells in culture, as well as using fresh tissue samples obtained from human or animal patients. Any of the foregoing provide a model to evaluate the effect of increasing intracellular sodium concentration on the proliferation and regeneration characteristics of these cells. Cell lines or primary samples can be obtain from virtually any tissue type, and thus a wide variety of tissue samples can be evaluated to see: (i) whether they endogenously express a voltage-gated sodium channel and (ii) what the pre-treatment intracellular sodium concentration is, and (iii) what the pre-treatment membrane potential is. This not only provides extensive understanding about how best to manipulate different types of cells, but may also provide a diagnostic criteria for evaluating when and how to manipulate intracellular sodium concentration to provide an efficacious effect. Additionally, these models provide an opportunity to evaluate dosage and timing parameters (e.g., what is the range of safe and effective doses; how long should tissue be treated).

In addition, proliferation and regeneration can be evaluated using a model system with a high innate level of regeneration potential, such as amphibians, zebrafish, and planarian. Of particular interest are studies designed to promote regeneration in these animals during a refractory period where the natural regeneration potential is very well. Promoting regeneration during the refractory period models promoting regeneration in an animal that does not have a naturally high level of regenerative potential.

Further, experiments can be performed in mammalian systems (e.g., systems in which the natural regenerative potential is limited). For example, the ear punch assay can be used. In this assay a hole is punched through the ear cartilage of a rodent. Methods and reagents can be evaluated based on their ability to promote regeneration of that tissue (e.g., promote regeneration of the complex cartilage and skin tissue—rather than just promote scab formation or skin closure). An additional example is the peripheral nerve transection model. Briefly, the sciatic nerve of a rodent (typically a rat or mouse) is transected. Methods and reagents can be evaluated based on their ability to restore all or partial function to the animal following transection.

A related animal model focuses on the spinal cord. An animal spinal cord can be cut (the sharp method) or compressed (the blunt method) to mimic various types of spinal cord injuries. The sharp method mimics an injury in which the spinal cord is actually partially or completed severed at a position. The blunt methods mimics an injury in which the spinal cord is compressed and considerable damage ensues due to swelling and bruising around the cord. Either model can be used to evaluate methods and reagents based on their ability to restore all or partial function to the animal following spinal cord injury.

Another animal model focuses on the digits. Small pieces of a rodent's tail and/or digits are amputated. The methods and reagents of the invention would be used and evaluated for their ability to promote regeneration of the amputated tail and digit tissue.

The foregoing is merely exemplary of some of the models that can be used.

Following extensive animal studies to ensure safety and efficacy, the methods and reagents are ultimately be tested on human patients.

The foregoing models are also useful for determining the optimal route of administration for a particular condition. For example, for determining whether an agent should be administered systemically, injected locally, perfused locally around the target tissue, and the like.

### VIII. Models of Cancer

Hyper-proliferative cells can be studied in vitro using transformed cell lines, cell lines made directly from patient tumor tissue, or using fresh samples of human or animal patient tissue. Any of the foregoing provide a model to evaluate the effect of decreasing intracellular sodium concentration on the growth and migration characteristics of these cells. Cell lines or primary samples can be obtained for virtually any tumor type, and thus a wide variety of cancer samples can be evaluated to see: (i) whether they endogenously express a voltage-gated sodium channel and (ii) what the pre-treatment intracellular sodium concentration is, and (iii) what the pre-treatment membrane potential is. This not only provides extensive understanding about which types of hyperproliferative cells are most likely to respond to a decrease in intracellular sodium concentration, but may also provide a diagnostic criteria for evaluating when decreasing intracellular sodium will likely be efficacious. Additionally, these models provide an opportunity to evaluate dosage and timing parameters (e.g., what is the range of safe and effective doses; how long should tissue be treated).

In addition to experiments conducted in cells and tissue samples in culture, numerous in vivo models exist. One model is referred to as the xenograft model whereby cancerous or pre-cancerous cells grown in culture are transplanted into an animal host. The animal host is then treated to treat the tumor. Such a model could easily be used here. Such a model would also allow one to evaluate whether the agent needs to be administered very locally (e.g., injected into the tumor or perfused around and through the tumor) or whether it can be administered systemically. Finally, animal models for specific types of cancers can be used in much the same way as described above for the xenograft model. Any in vivo animal model would allow an evaluation of the tolerability and efficacy of the treatment, particularly whether both tumor growth and migration are effected equally or whether the treatment has a more pronounced effect on one over the other.

Furthermore, we note that studies conducted across a range of different types of hyper-proliferative conditions (e.g., various solid tumors, blood cancers, etc.) will allow for rational selection of treatments based on the likelihood the treatment will be effective for that hyper-proliferative condition.

The foregoing is merely exemplary of the cell and animal models that can be used to further evaluate the efficacy of the compounds and methods of the invention.

The foregoing models are also useful for determining the optimal route of administration for a particular condition. For example, for determining whether an agent should be administered systemically, injected locally, perfused locally around the target tissue, and the like.

### IX. Administration

For any of the foregoing classes of compounds that can be used in the various methods of the present invention, one of skill in the art can select amongst available delivery methods to administer the compound to the particular cells, tissue, or organs in vitro, in vivo, or ex vivo. The term “administering” is used to refer to providing a compound to cells, tissues, organs, or structures in vitro, in vivo, or ex vivo. By way of example, many compounds readily transit epidermal barriers and other biological membranes. To administer such compounds to cells or to an animal, the compound can simply be dissolved and added to the fluid in which the cells or animal is cultured. Alternatively, the compound can be dissolved and added to the animals food or drinking water. In another alternative, the compound can be administered to the animal via local or systemic injection.

Certain compounds do not as readily transit epidermal barriers and biological membranes, and thus additional techniques have been adapted to administer such compounds to cells, tissues, and organisms. For example, RNAi constructs are often administered to animals by addition to their food or drinking water. Numerous types of nucleic acids are delivered via viral or plasmid-based expression vectors. Polypeptide-based compounds that do not readily transit membrane or that are not actively transported into cells via receptor-mediated mechanisms can be administered along with carriers that facilitate transit into cells and tissues. The foregoing exemplary administration methods are well known in the art and can be selected based on the compounds and organisms being employed in the particular methods of use.

Whether compounds are being administered as part of an assay or as part of a method for modulating cell behavior, compounds can be administered alone or as pharmaceutical formulations. Exemplary pharmaceutical compositions are formulated for administration to cells or animals. In certain embodiments, the compound included in the pharmaceutical preparation may be active itself, or may be a prodrug, e.g., capable of being converted to an active compound in a physiological setting. In certain embodiments the subject compounds may be simply dissolved or suspended in water, for example, in sterile water. In certain embodiments, the pharmaceutical preparation is non-pyrogenic, i.e., does not elevate the body temperature of an animal.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a subject compound. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments the agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Another aspect of the present invention provides pharmaceutically acceptable compositions comprising an effective amount of one or more of the compounds described herein, formulated together with one or more pharmaceutically acceptable carriers (additives) and/or diluents. The pharmaceutical compositions of the present invention may be specially formulated for administration in solid or liquid form, including, but not limited to, those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; wound dressing applied directly to a site of injury or wound; or (4) intravaginally or intrarectally, for example, as a pessary, cream or foam; or (5) opthalamic administration, for example, for administration following injury or damage to the retina; (6) administration to the spinal cord to promote regeneration of the structure, in part or whole, via a continuous infusion that bathes the spinal cord; (7) by local injection or perfusion of the target tissue; or (8) administration to a severed digit or limb via a sleeve containing the composition. However, in certain embodiments the subject compounds may be simply dissolved or suspended in sterile water. In certain embodiments, the pharmaceutical preparation is non-pyrogenic, i.e., does not elevate the body temperature of a human or animal patient.

### EXEMPLIFICATION

The invention now being generally described, it will be more readily understood by reference to the following examples, which are included merely for purposes of illustration of certain aspects and embodiments of the present invention, and are not intended to limit the invention. For example, the particular experimental design disclosed herein represent exemplary tools and methods for validating proper function. As such, it will be readily apparent that any of the disclosed specific experimental plan can be substituted within the scope of the present disclosure.

### Example 1

### Na_{V}1.2 is Specifically Expressed During Early Regeneration But Not Wound Healing

Voltage-gated sodium channels (VGSCs) are plasma membrane proteins that regulate sodium influx into cells (Yu and Catterall, 2003), and two are known in Xenopus: Na_{V}1.2 and Na_{V}1.5. To assess whether these voltage-gated sodium channels might play a role during tail regeneration, we examined their expression in the regeneration bud. While Na_{V}1.5 is not expressed (data not shown), Na_{V}1.2 protein becomes expressed by 18 hpa (FIGS. 1A and 1B) and persists until 2 days post amputation (dpa) (FIG. 1C). This expression pattern is consistent with a role for Na_{V}1.2 in tail regeneration.

Interestingly, Na_{V}1.2 protein was detected only in the mesenchymal cells of the regeneration bud (FIG. 1D, red arrow), unlike V-ATPase, a key ion transporter for regeneration which is also expressed in the wound epithelium (Adams et al., 2007). To determine whether Na_{V}1.2 may also function in wound healing, we examined its expression in flank wounds at 24 hours. In contrast to V-ATPase, which is up-regulated in such wounds (FIG. 1E), Na_{V}1.2 protein was not detected at the wound edge (FIG. 1F). Thus, Na_{V}1.2 is a unique molecular marker that specifically distinguishes true regeneration (a process that restores the complex caudal structure after amputation) from simple wound healing.

### Example 2

### Pharmacological Inhibition of Na_{V}1.2 Activity Prevents Tail Regeneration in Xenopus Larvae

To determine if Na_{V}1.2 activity is required for tail regeneration, we ablated its function molecularly by RNA interference (RNAi) using a plasmid encoding a short RNA hairpin construct specifically targeting the Xenopus Na_{V}1.2 gene. The targeting vector carries a GFP marker (Miskevich et al., 2006), enabling the selection of animals that expressed the construct in the distal tail (FIGS. 1G and 1H). Expression of the Na_{V}1.2 short RNAi hairpin in the tail region at the site of amputation (yellow arrows) efficiently inhibited tail regeneration (Regeneration Index=RI, see “Tail Regeneration Assay” in Methods; RI=198, n=100, versus RI=261, n=72 for control RNAi targeting dsRED, a fluorescent protein that is not endogenous to Xenopus, p<0.01), demonstrating that Na_{V}1.2 is specifically required for tail regeneration (FIG. 1H).

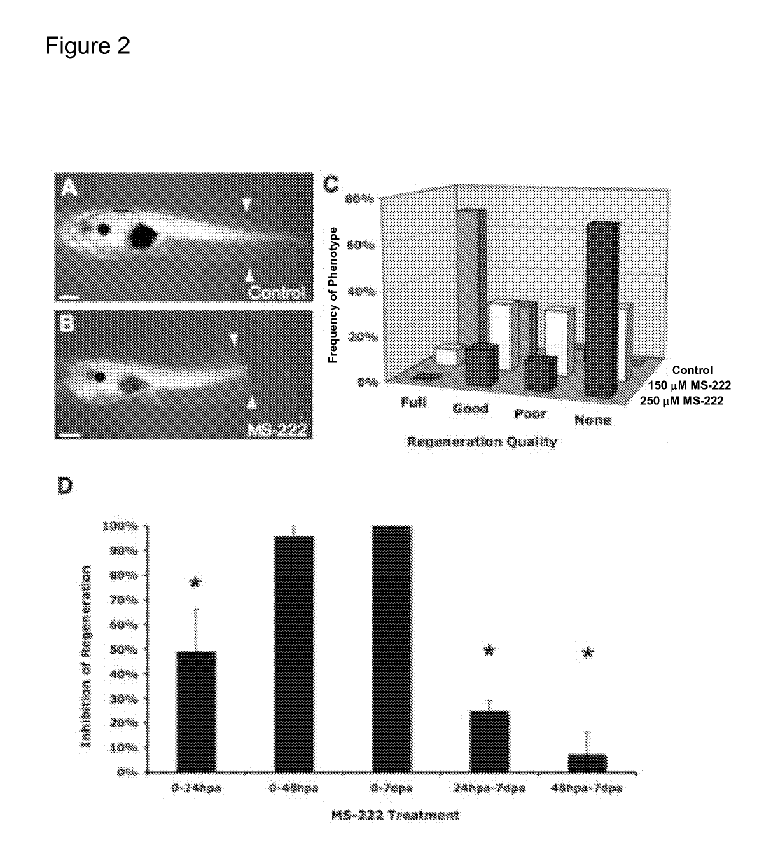

To characterize the temporal requirement of Na_{V}1.2 function during regeneration, we looked to utilize pharmacological inhibitors of Na_{V}1.2 that will enable us to vary the timing of the functional blockade. First, we determined whether chemical inhibition of Na_{V}1.2 activity recapitulates its loss-of-function phenotype during regeneration. MS-222 (also known as tricaine) is a well-known inhibitor of VGSCs (Hedrick and Winmill, 2003) that blocks inward Na⁺ currents (Frazier and Narahashi, 1975). Treatment of animals with MS-222 immediately after tail amputation significantly inhibited regenerative ability (RI=62, n=100) (FIG. 2B) when compared to controls (RI=265, n=97, p<0.01) (FIG. 2A), and in a dose-dependent manner (FIG. 2C). The treatment did not induce significant apoptosis in the bud (data not shown). Importantly, the μM concentrations used for the tail assays were approximately ten-fold lower than what is usually used for amphibian anesthesia (generally in the mM range). Similar to the Na_{V}1.2 RNAi experiment, the overall development and movement of the animals were normal during pharmacological treatment, and had no effect on primary tail formation during early embryogenesis. We conclude that Na_{V}1.2 function is specifically required for regeneration, and that both pharmacological and molecular loss-of-function to selectively reveal the importance of Na_{V}1.2.

### Example 3

### Na_{V}1.2 Activity is Required During Regeneration

To examine the temporal requirement for Na_{V}1.2 function during this process, we amputated tails of tadpoles, treated them with MS-222 for specific durations, and assayed their regenerative ability. Treatment with MS-222 through the entire length (7 days) of the assay was sufficient to inhibit regeneration in 82% of the tails (FIG. 2D); likely this effect is stronger than that of the RNAi due to the mosaicism observed with DNA injections (FIG. 1H). Blocking Na_{V}1.2 activity for the first 24 hours post amputation prevented tail regeneration in 45% of the tadpoles, indicating that Na_{V}1.2 plays a role in bud establishment. When the duration of MS-222 treatment was expanded to include the first 48 hpa, 78% of the tails failed to regenerate, fully recapitulating the severity of phenotype seen when Na_{V}1.2 function was inhibited throughout the full 7 days required for normal regeneration. However, addition of MS-222 to block Na_{V}1.2 activity at 48 hpa, after regenerative outgrowth has begun, had no effect on inhibiting regeneration. These results strongly suggest that Na_{V}1.2 function is principally required during the establishment and early outgrowth phases of regeneration.

### Example 4

### Inhibition of Na_{V}1.2 Activity Abolishes Sodium Flux During Regeneration

Na_{V}1.2 is a membrane-bound Na⁺ transporter that regulates the influx of Na⁺ into cells (Yu and Catterall, 2003). Thus, we examined the functional consequences of Na⁺ transport in regeneration using MS-222. To visualize Na⁺ flux, we used CoroNa Green, a fluorescent Na⁺ indicator dye that selectively interacts with Na⁺ ions and exhibits an increase in fluorescent emission upon binding (Meier et al., 2006). In intact tails, a few randomly distributed cells appear positive for the CoroNa Green signal relative to the majority of the tail population (FIG. 3A). At 24 hpa, a specific CoroNa Green signal is strong in the regeneration bud region but not in the rest of the tail and trunk (FIG. 3B), suggesting a significant increase in Na⁺ transport into the cells of the bud during normal regeneration. This observation correlates with Na_{V}1.2 being specifically up-regulated in the bud after amputation. When the amputated tails were treated with the Na_{V} inhibitor, MS-222, the CoroNa Green signal was abolished (FIG. 3C, p<0.02), confirming that inhibition of Na_{V}1.2 function abrogates Na⁺ influx into the bud. Given that MS-222 inhibits regeneration, the observed control of Na⁺ content by Na_{V}1.2 indicates that this channel functions in regeneration principally through its modulation of Na⁺ flux into bud cells.

### Example 5

### Inhibition of Na_{V}1.2 Function Reduces Proliferation and Alters Axonal Migration

To understand the cellular basis for regenerative failure in animals with loss of Na_{V}1.2 function, we examined proliferation in MS-222-treated amputated tails. During development, mitotic cells are observed to be randomly located in the growing tail. In normal regeneration, an increased number of proliferating cells are concentrated at the regeneration bud by 48 hpa (Adams et al., 2007). We quantified the number and distribution of proliferating cells in regenerating tails at 48 hpa using an antibody to phosphorylated Histone 3B (H3P), a marker of the G₂/M transition of the cell cycle that identifies mitotic cells in Xenopus and many other systems (Adams et al., 2007; Saka and Smith, 2001). Inhibition of Na_{V}1.2 function by MS-222 resulted in a 90% decrease in the number of mitotic cells in the regeneration bud region (1.3±1.5, n=4) as compared to control siblings (12.5±4.8, n=4, p<0.005) (FIG. 3D). Many H3P-positive cells are seen in the wild-type regeneration bud (FIG. 3E) but very few are detected in MS-222 treated buds (FIG. 3F). In contrast, no significant change in proliferation was seen in the central tail “flank” region (71±27 H3P-positive cells for control as compared to 66.3±10 H3P-positive cells for MS-222 treatment, n=8, p=0.38) (FIG. 3D′), suggesting that Na_{V}1.2 activity is not a general requirement for normal cell division. Together, these data demonstrate that Na_{V}1.2 is necessary for the specific up-regulation of proliferation in the regenerative growth region during endogenous tail regeneration.

It has long been known that regenerative growth requires proper innervation (Singer, 1952, 1965). Thus, we asked whether Na_{V}1.2 regulates neural regeneration by examining the neuronal pattern in the amputated tail of tadpoles treated with MS-222. In normal 3-day old tail regenerates, axons appear in bundles that grow and concentrate to the end of the regenerate, in a direction parallel to the anterior-posterior axis (FIG. 3G). In contrast, chemical inhibition of Na_{V}1.2 caused axons to extend circumferentially along the edge of the regeneration bud, perpendicular to the main axis of tail growth (FIG. 3H). Notably, the overall quantity of neurons appeared to be reduced as compared to its control siblings. These results suggest that Na_{V}1.2 is required for proper innervation of the regenerate.

### Example 6

### Na_{V}1.2 Expression is Regulated by Membrane Potential

In order to better understand the role of Na_{V}1.2, we examined its potential relationships with known regenerative pathways. First, we investigated the pathways potentially regulating Na_{V}1.2 expression. Inhibition of Na_{V}1.2 in amputated tails did not alter V-ATPase expression in the 24 hpa regeneration bud (data not shown). However, in amputated tails treated with Concanamycin, a specific inhibitor of V-ATPase activity (Huss et al., 2002) that blocks regeneration (Adams et al., 2007), the expression of Na_{V}1.2 protein was absent at 24 and 48 hpa (FIGS. 4A-4C). Thus, our results indicate that Na_{V}1.2 function is dependent upon V-ATPase function but not vice versa. We next examined Na_{V}1.2 protein expression during the refractory stages, an endogenous period during development in which tadpoles temporarily lose the ability of caudal regeneration (Beck et al., 2003). Na_{V}1.2 protein was not detected in 24-hour tail stumps amputated during this non-regenerative state (FIG. 4D). Thus, the presence or absence of Na_{V}1.2 is predictive of regenerative ability, consistent with the above-demonstrated functional roles.

Since V-ATPase controls the cell membrane voltage potential in the regeneration bud (Adams et al., 2007), the endogenous up-regulation of Na_{V}1.2 at 18 hpa could be attributed to either H′-pump-mediated changes in the transmembrane potential of bud cells, or to other unknown functions of the V-ATPase protein complex. To distinguish between these possibilities, we used palytoxin, which converts ubiquitous Na⁺-potassium transporters into non-specific pores (Tosteson et al., 2003) to depolarize regeneration bud cells while maintaining normal V-ATPase activity. Exposure of amputated tails to 2 nM palytoxin significantly reduced regeneration without affecting overall development, growth, or V-ATPase expression (Adams et al., 2007). In the presence of palytoxin, Na_{V}1.2 protein was not detected in the regeneration bud at 24 hpa (FIG. 4E), suggesting that the endogenous induction of Na_{V}1.2 expression by V-ATPase function occurs through the H⁺ pump's control of transmembrane potential of regeneration bud cells.

### Example 7

### Inhibition of Na_{V}1.2 Function Reduces Expression of Downstream Genes that Drive Regenerative Outgrowth

Several pathways have been shown to be required for driving regenerative outgrowth and patterning in the tail, including Notch, Msx1, and BMP (Beck et al., 2006; Beck et al., 2003; Sugiura et al., 2004). RNA expression of Notch, Msx1, and BMP components can be observed after 24 hpa in the regenerating tail tissues. We performed in situ hybridization using gene-specific RNA probes to examine the expression patterns of Notch and Msx1 in the regeneration bud after treatment with MS-222. In wild-type regeneration buds at 48 hpa, Notch1 is normally expressed in the neural ampulla and in the mesenchymal region of the regeneration bud (FIGS. 4F and 4H). In contrast, Na_{V}1.2-inhibited tail buds exhibited greatly reduced levels of Notch1 that was mislocalized to the tip (FIGS. 4G and 4I). At 48 hpa, Msx1 was expressed in the neural ampulla and at the epithelial edge of the regenerating tail tip (FIGS. 4J and 4L). When Na_{V} activity was blocked, Msx1 expression was abolished (FIGS. 4K and 4M). Likewise, levels of BMP2, BMP4, and Delta were greatly reduced in the presence of MS-222 (data not shown). These results demonstrate that Na_{V}1.2 acts upstream to regulate the expression of several key genes that are known to control caudal regenerative outgrowth and patterning.

### Example 8

### Na_{V}1.2 Controls Regeneration Through Modulation of Intracellular Na⁺ Levels, not Vₘₑₘ

To gain a detailed mechanistic understanding of Na_{V}1.2 activity, we examined the consequences of modulating Na⁺ ion transport in the regenerating tail. During development, the intact tail is hyper-polarized with a few depolarized cells that are randomly distributed (Adams et al., 2007). However, after tail amputation, the presumptive regeneration bud becomes highly depolarized. V-ATPase is active by 6 hpa and its function is required to re-polarize the bud by 24 hpa. If the bud is polarized at this timepoint, then regeneration proceeds normally; if the bud remains depolarized (as when V-ATPase is inactivated, either during the refractory period or by Concanamycin), then regeneration fails. Because VGSC activity is also a well-known major determinant of a cell's membrane potential level, we hypothesized that loss of Na_{V}1.2 activity could alter the membrane voltage state of the regeneration buds at 24 hpa. Using the voltage-reporter dye bis-(1,3-dibutylbarbituric acid) pentamethine oxonol (DiBAC₄(3)) (Epps et al., 1994), we did not observe any changes in membrane voltage in the regeneration bud cells of MS-222 treated tails as compared to control (FIGS. 4O and 4P, regeneration bud area outlined in red), suggesting that changes in transmembrane potential are not the mechanism by which Na_{V}1.2 function controls regenerative behavior.

Since Na_{V}1.2 controls Na⁺ levels in the regeneration bud (FIGS. 3B and 3C), we next tested the hypothesis that modulation of intracellular Na⁺ concentration (normally mediated by Na_{V}1.2) may be the direct biophysical signal that induces downstream pathways. Even though Na⁺ currents mostly do not act as secondary messengers, a known effector of Na⁺ ion-based signaling is the salt-inducible kinase (SIK1), a member of the AMP-activated protein kinase (PKA) family and a Class II HDAC kinase that responds to changes in intracellular Na⁺ levels (Sanz, 2003). SIK1 is thus a potential candidate for a molecular sensor of Na_{V}1.2 activity during regeneration. Staurosporine (STS) is a Serine/Theronine kinase inhibitor that directly binds SIK1 (Katoh et al., 2006). Treatment of amputated tails with 10 nM STS significantly inhibited tail regeneration, reducing the regenerative ability to 28% of controls (control RI=226, STS-treated RI=64, n=52, p<0.001) (FIG. 4Q). Development of the treated animals was otherwise unaffected. This result suggests SIK1 could act as a specific effector that transduces physiological Na_{V}1.2 activity into second messenger cascades that can control some of the important functions necessary for tail regeneration.

### Example 9

### Transient Induction of Na⁺ Current During the Refractory Period is Sufficient to Induce Regeneration

Mammals exhibit an age-dependent decrease in regenerative potential (Illingworth, 1974). Similarly, Xenopus also show a reduction of caudal regenerative potential during the refractory period (Beck et al., 2003). Thus it is an excellent context to identify the key differences between permissible and non-permissible regenerative states. Tails amputated during the refractory period show a notably thickened, non-regenerative wound epidermis (WE) by 24 hpa (Beck et al., 2003). Consistent with the observation that amputations of tails at non-regenerative stages exhibit altered healing, 18 hpa refractory caudal stumps have a thickened WE (FIG. 5A (red arrows bracketing WE) and 5A′-width of epidermis outlined by dashed red line) compared to a regenerative bud WE at the same timepoint (FIGS. 5B and 5B′-dashed red line indicates epidermis width). This observation suggests that at 18 hpa, refractory buds have already completed non-regenerative wound healing. Knowing that Na_{V}1.2 is strongly expressed by 18 hpa in the normal regeneration bud, but not in amputated refractory tails (FIG. 4D), we hypothesized that induction of Na⁺ current mediated by Na_{V}1.2 in the refractory tail bud at 18 hpa could be sufficient to promote regeneration.

Although mis-expression of ion transporters is an important technique for regulating regenerative ability (Adams et al., 2007), our data suggest a direct role for sodium ions in this process. Thus, we identified a pharmacological method that would allow us to modulate sodium transport temporally. Monensin is an ionophore that selectively transports Na⁺ ions into cells (Mollenhauer et al., 1990). Tails of animals in the refractory stage were amputated and at 18 hpa, treated with 20 uM monensin in a medium containing 90 mM Na⁺ (normal culture medium contains 10 mM Na⁺). To confirm that monensin induces an increase in intracellular Na⁺ levels, we used the CoroNa Green indicator dye to visualize Na⁺ content in the amputated caudal region. At 19 hpa (after an one-hour current induction), normal refractory tail buds show very weak CoroNa Green signal (FIG. 5C). In contrast, monensin-treated tails in high Na⁺ medium showed a strong CoroNa Green fluorescence at the amputation site (FIG. 5D), demonstrating that this treatment increases intracellular Na⁺ content in the regeneration bud. We then assessed the consequence of monensin treatment on regeneration. During the refractory period, tail regenerative ability of Xenopus tadpoles is extremely poor (R=11.5, n=62). Most animals (89%) failed to regenerate any tissue in the amputated tails while a small percentage regenerate poorly (FIG. 5E). Strikingly, treatment 18 hpa with 20 uM monensin in a medium containing 90 mM Na⁺ for just one hour induced a significant increase in regenerative ability (RI=59.1, n=44, p<0.005) compared to refractory controls (FIG. 5F). Importantly, the same treatment with either monensin alone or high extracellular Na⁺ alone did not improve regenerative ability (data not shown), showing that neither monensin nor just high extracellular sodium alone are capable of this effect: it is the forced sodium influx that results in regeneration. The sufficiency of a brief transient pulse of Na⁺ current at 18 hpa to rescue regeneration in the amputated refractory tails demonstrates that it is principally the change in the intracellular Na⁺ level in the regeneration bud cells that mediates downstream regenerative pathways (and not any non-conducting role of Na_{V}1.2). Most importantly, this instructive signal does not have to be present at the time of injury and is not required long term to drive complete regeneration.

### Discussion

The present disclosure summarizes experiments that revealed a novel role for intracellular sodium in controlling regeneration. As a example, tail regeneration in Xenopus is modulated, at least in part, by intracellular sodium concentration regulated via the voltage-gated sodium channel Na_{V}1.2.

The experiments detailed herein provide, in part, a model for the endogenous pathway that drives tail regeneration in Xenopus. However, these experiments also show that, although regeneration-essential increases in intracellular sodium concentration are endogenously mediated by NaV1.2 in this system, intracellular sodium concentration can also be manipulated using pharmacological reagents—including reagents that permit modulation of intracellular sodium concentration without relying on expression of a voltage-gated sodium channel.

### Methods

The above experiments were conducted using the following methods.

### Tail Regeneration Assay

Xenopus laevis larvae were cultured using standard protocols approved for the care of experimental organisms. Tails at stages 40-41, or stages 45-47 (refractory period) (Nieuwkoop and Faber, 1967) were amputated under a dissecting microscope using a scalpel blade at the midpoint between the anus and the tip. Tadpoles were cultured in (0.1×MMR±inhibitor) at 22° C. for 7 days and scored for tail regeneration. Drug experiments were carried out at least in duplicate.

To quantify and compare regeneration efficiency of tadpoles treated with different reagents, the “Regeneration Index” (RI) was determined. The RI evaluates the efficiency of regeneration for each treatment and allows for comparison of the effect of various treatments to controls.

Individual animals for each specific treatment were each scored as follows:

Full: complete regeneration (indistinguishable from uncut controls).

Good: robust regeneration with minor defects (e.g., missing fin, curved axis).

Poor: poor regeneration (hypomorphic/defective regenerates).

None: no regeneration

For each treatment, the percentage of regenerates belonging to each category were calculated, and then multiplied by 3, 2, 1 or 0, respectively for, Full, Good, Poor and None. The resulting R1 for each condition tested ranges from 0 to 300, with 0 corresponding to no regeneration, and 300 for complete regeneration.

Xenopus laevis larvae were cultured via approved protocols (IACUC #M2008-08). Tails at stages 40-41, or stages 45-47 (refractory period) (Nieuwkoop and Faber, 1967) were amputated at the midpoint between the anus and the tip. Tadpoles were cultured in (0.1×MMR±reagent) at 22° C. for 7 days and scored for tail regeneration. To quantify and compare regeneration in groups of tadpoles treated with different reagents, we determine the “Regeneration Index” (RI), ranging from 0 (no regeneration) to 300 (complete regeneration) as previously described in (Adams et al., 2007).

### RNA Interference

DNA oligos encoding short RNA hairpins (shRNA) targeting Xenopus Na_{V}1.2 (GenBank Accession No. AY121368) or dsRED (GenBank Accession No. AY679106; Discosoma sp. RC-2004 red fluorescent protein R1 mRNA) were cloned into the multiple cloning site of a modified pcDNA3.1 vector downstream of a U6 RNA Pol III promoter, and also containing a CMV-driven GFP marker (Miskevich et al., 2006) (gift of F. Miskevich). For the sense strand, the Na_{V}1.2 (@391 bp) RNAi target sequence is: 5′-GCCATGGAGCATTATCCAATG-3′ and the dsRED (@597 bp) RNAi target sequence is: 5′-GTTCAAGTCCATCTACATGGC-3′. The @ designation indicates where along the nucleic acid target sequence the RNAi construct begins. These constructs were micro-injected into 1 or 2-cell stage embryos. The presence of the shRNA in st.40 tail tissues was identified by the expression of GFP using fluorescence microscopy.

### Modulation and Imaging of Sodium Flux

At 23 hpa, tadpoles were incubated in 90 μM of CoroNa Green indicator dye (Invitrogen) in 0.1×MMR for 45 minutes and washed twice in 0.1×MMR+30 uM BTS (to immobilize tadpole movement). At 24 hpa, the CoroNa Green signal was excited at 488 nM and fluorescence emission data was collected at 592 nM. Data was analyzed using IPLab software. For induction of Na⁺ current, 0.1×MMR was supplemented with sodium gluconate (Sigma) to increase the Na⁺ concentration to 90 mM. For refractory period analysis, tails were amputated at st. 47, at 18 hpa, and animals were treated with or without 90 mM Na⁺ and 20 μM monensin (Sigma) in 0.1×MMR with 90 μM CoroNa Green for 45 minutes and washed twice in 0.1×MMR+50 uM BTS. Imaging of Vₘₑₘ using DiBAC₄(3) (Invitrogen) was performed exactly as described in (Adams et al., 2007).

### In Situ Hybridization

Embryos were fixed in MEMFA (Sive et al., 2000) and dehydrated in methanol. In situ hybridization was carried out according to standard protocols (Harland, 1991) with probes to: Na_{V}1.2 (AY121368), Na_{V}1.5 (Armisen et al., 2002), Notch1 (Coffman et al., 1990), and Msx1 (Feledy et al., 1999).

### Immunohistochemistry

Xenopus embryos were fixed overnight in MEMFA, heated for 2 hrs at 65° C. in 50% formamide (to inactivate endogenous alkaline phosphatases), permeabilized in PBTr+0.1% Triton X100 for 30 min, and processed for immunohistochemistry using alkaline phosphatase secondary antibody (Levin, 2004) until signal was optimal and background minimal (usually 12 hrs). The expression profiles represent consensus patterns obtained from the analysis of 8-12 embryos at each stage. Pan-anti-NaV (Sigma S-8809), anti-acetylated a-tubulin (Sigma #T6793), and anti-phospho-H3 (Upstate #05-598) antibodies were used at 1:1000.

### REFERENCES

Adams, D., et al., 2006. Xenopus tadpole tail regeneration requires the activity of the proton pump V-ATPase, and proton pumping is sufficient to partially rescue the loss-of-function phenotype. Developmental Biology. 295, 355-356.
Adams, D. S., et al., 2007. H+ pump-dependent changes in membrane voltage are an early mechanism necessary and sufficient to induce Xenopus tail regeneration. Development. 134, 1323-35.
Beck, C. W., et al., 2006. Temporal requirement for bone morphogenetic proteins in regeneration of the tail and limb of Xenopus tadpoles. Mech Dev. 123, 674-88.
Beck, C. W., et al., 2003. Molecular pathways needed for regeneration of spinal cord and muscle in a vertebrate. Dev Cell. 5, 429-39.
Beck, C. W., et al., 2009. Beyond early development: Xenopus as an emerging model for the study of regenerative mechanisms. Dev Dyn.
Birnbaum, K. D., Alvarado, A. S., 2008. Slicing across kingdoms: regeneration in plants and animals. Cell. 132, 697-710.
Brackenbury, W. J., Djamgoz, M. B., 2006. Activity-dependent regulation of voltage-gated Na⁺ channel expression in Mat-LyLu rat prostate cancer cell line. J. Physiol. 573, 343-56.
Chen, Y., et al., 2006. Control of muscle regeneration in the Xenopus tadpole tail by Pax7. Development. 133, 2303-13.
Ding, Y., Djamgoz, M. B., 2004. Serum concentration modifies amplitude and kinetics of voltage-gated Na⁺ current in the Mat-LyLu cell line of rat prostate cancer. Int J Biochem Cell Biol. 36, 1249-60.
Diss, J. K., et al., 2005. A potential novel marker for human prostate cancer: voltage-gated sodium channel expression in vivo. Prostate Cancer Prostatic Dis. 8, 266-73.
Djamgoz, M. B. A., et al., 2001. Directional movement of rat prostate cancer cells in direct-current electric field: involvement of voltage-gated Na⁺ channel activity. Journal of Cell Science. 114, 2697-705.
Epps, D., et al., 1994. Characterization of the steady-state and dynamic fluorescence properties of the potential-sensitive dye bis-(1,3-dibutylbarbituric acid)trimethine oxonol (Dibac4(3)) in model systems and cells. Chem. Phys. Lipids. 69, 137-150.
Fraser, S. P., et al., 2005. Voltage-gated sodium channel expression and potentiation of human breast cancer metastasis. Clin Cancer Res. 11, 5381-9.
Fraser, S. P., et al., 2004. T-lymphocyte invasiveness: control by voltage-gated Na⁺ channel activity. FEBS Lett. 569, 191-4.
Fraser, S. P., et al., 2000. Effects of voltage-gated ion channel modulators on rat prostatic cancer cell proliferation: comparison of strongly and weakly metastatic cell lines. Prostate. 44, 61-76.
Gardiner, D. M., 2005. Ontogenetic decline of regenerative ability and the stimulation of human regeneration. Rejuvenation Res. 8, 141-53.
Gargioli, C., Slack, J. M., 2004. Cell lineage tracing during Xenopus tail regeneration. Development. 131, 2669-79.
Harland, R. M., In situ hybridization: an improved whole mount method for Xenopus embryos. In: B. K. Kay, H. B. Peng, Eds.), Xenopus laevis: Practical uses in cell and molecular biology, Vol. 36. Academic Press, San Diego, 1991, pp. 685-695.
Hedrick, M. S., Winmill, R. E., 2003. Excitatory and inhibitory effects of tricaine (MS-222) on fictive breathing in isolated bullfrog brain stem. Am J Physiol Regul Integr Comp Physiol. 284, R405-12.
Ho, D. M., Whitman, M., 2008. TGF-beta signaling is required for multiple processes during Xenopus tail regeneration. Dev Biol. 315, 203-16.
Huss, M., et al., 2002. Concanamycin A, the specific inhibitor of V-ATPases, binds to the Vo subunit c. Journal of Biological Chemistry. 277, 40544-8.
Ingber, D. E., Levin, M., 2007. What lies at the interface of regenerative medicine and developmental biology? Development. 134, 2541-7.
Isbilen, B., et al., 2006. Docosahexaenoic acid (omega-3) blocks voltage-gated sodium channel activity and migration of MDA-MB-231 human breast cancer cells. Int J Biochem Cell Biol. 38, 2173-82.
Katoh, Y., et al., 2006. Silencing the constitutive active transcription factor CREB by the LKB1-SIK signaling cascade. FEBS J. 273, 2730-48.
Levin, M., 2004. A novel immunohistochemical method for evaluation of antibody specificity and detection of labile targets in biological tissue. Journal of Biochemical and Biophysical Methods. 58, 85-96.
Levin, M., 2006. Is the early left-right axis like a plant, a kidney, or a neuron? The integration of physiological signals in embryonic asymmetry. Birth Defects Res C Embryo Today. 78, 191-223.
Levin, M., 2007. Large-scale biophysics: ion flows and regeneration. Trends in Cell Biology. 17, 262-271.
Levin, M., 2009. Bioelectric mechanisms in regeneration: Unique aspects and future perspectives. Semin Cell Dev Biol.
Meier, S. D., et al., 2006. Properties of the new fluorescent Na+ indicator CoroNa Green: comparison with SBFI and confocal Na⁺ imaging. J Neurosci Methods. 155, 251-9.
Miskevich, F., et al., 2006. RNA interference of Xenopus NMDAR NR1 in vitro and in vivo. J Neurosci Methods. 152, 65-73.
Mochii, M., et al., 2007. Tail regeneration in the Xenopus tadpole. Dev Growth Differ. 49, 155-61.
Mollenhauer, H. H., et al., 1990. Alteration of intracellular traffic by monensin; mechanism, specificity and relationship to toxicity. Biochim Biophys Acta. 1031, 225-46.
Mycielska, M. E., Djamgoz, M. B., 2004. Cellular mechanisms of direct-current electric field effects: galvanotaxis and metastatic disease. J Cell Sci. 117, 1631-9.
Nieuwkoop, P. D., Faber, J., 1967. Normal Table of Xenopus laevis (Daudin). North-Holland Publishing Company, Amsterdam.
Nuccitelli, R., et al., 1986. On electrical currents in development. Bioessays. 5, 292-294.
Onganer, P. U., Djamgoz, M. B., 2005. Small-cell lung cancer (human): potentiation of endocytic membrane activity by voltage-gated Na(+) channel expression in vitro. J Membr Biol. 204, 67-75.
Onganer, P. U., et al., 2005. Neuronal characteristics of small-cell lung cancer. Br J Cancer. 93, 1197-201.
Poss, K. D., et al., 2000. Roles for Fgf signaling during zebrafish fin regeneration. Developmental Biology. 222, 347-58.
Poss, K. D., et al., 2002. Heart regeneration in zebrafish. Science. 298, 2188-90.
Saka, Y., Smith, J. C., 2001. Spatial and temporal patterns of cell division during early Xenopus embryogenesis. Dev Biol. 229, 307-18.
Sanz, P., 2003. Snfl protein kinase: a key player in the response to cellular stress in yeast. Biochem Soc Trans. 31, 178-81.
Singer, M., 1952. The influence of the nerve in regeneration of the amphibian extremity. Q Rev Biol. 27, 169-200.
Singer, M., A theory of the trophic nervous control of amphibian limb regeneration, including a re-evaluation of quantiative nerve requirements. Proceedings of Regeneration in Animals, 1965.
Sive, H. L., et al., 2000. Early Development of Xenopus Laevis. Cold Spring Harbor Laboratory Press, New York.
Slack, J. M., 2003. Regeneration research today. Dev Dyn. 226, 162-6. Stocum, D. L., Zupanc, G. K., 2008. Stretching the limits: stem cells in regeneration science. Dev Dyn. 237, 3648-71.
Tosteson, M. T., et al., 2003. Effects of palytoxin on cation occlusion and phosphorylation of the (Na+,K+)-ATPase. J Membr Biol. 192, 181-9.
Tseng, A. S., Levin, M., 2008. Tail regeneration in Xenopus laevis as a model for understanding tissue repair. J Dent Res. 87, 806-16.
Yokoyama, H., 2008. Initiation of limb regeneration: the critical steps for regenerative capacity. Dev Growth Differ. 50, 13-22.

### INCORPORATION BY REFERENCE

All publications and patents mentioned herein, are hereby incorporated by reference in their entirety as if each individual publication or patent was specifically and individually indicated to be incorporated by reference.

1. A method of promoting one or more of proliferation or differentiation, comprising contacting a cell culture with an effective amount of an agent to increase intracellular sodium concentration in cells of said cell culture, wherein said agent is selected from a sodium ionophore, or insulin, or both, and wherein the agent induces Na⁺ influx into said cell, thereby promoting one or more of proliferation or differentiation.

2. A method of promoting tissue regeneration, comprising contacting a cell culture with an effective amount of an agent to increase intracellular sodium concentration in cells of said cell culture, wherein said agent is selected from a sodium ionophore, or insulin, or both, and wherein the agent induces Na⁺ influx into said cell, thereby promoting tissue regeneration.

3. A method for promoting one or more of proliferation or differentiation, comprising administering an effective amount of an agent, wherein said agent is selected from a sodium ionophore, or insulin, or both, and wherein the agent induces Na⁺ influx into cells, thereby promoting one or more of proliferation or differentiation.

4. A method for promoting tissue regeneration, comprising administering an effective amount of an agent, wherein said agent is selected from a sodium ionophore, or insulin, or both, and wherein the agent induces Na⁺ influx into said cells, thereby promoting tissue regeneration.

5. The method of claim 4, wherein said sodium ionophore is monensin.

6. The method of claim 5, wherein said Na⁺ influx does not alter the membrane potential of said cells.

7. The method of claim 5, wherein said method or use promotes regeneration of an appendage or organ.

8. The method of claim 5, wherein said method or use promotes regeneration of one or more of muscle tissue and neuronal tissue.

9. The method of claim 1, wherein the cells comprise progenitor cells.

10. The method of claim 9, wherein said progenitor cell is selected from one or more of an embryonic stem cell, a neural progenitor cell, a neural crest progenitor cell, a mesenchymal stem cell, or a muscle progenitor cell.

11. The method of claim 9, wherein, prior to contact with said agent, the cell culture comprises a medium having a higher sodium concentration relative to the intracellular sodium concentration of the cell.

12. The method of claim 1, wherein, prior to contact with said agent, the cells are in a non-proliferative state.

13. The method of claim 12, wherein said agent induces Na⁺ influx into said cells via an endogenously expressed voltage-gated sodium channel.

14. A method for inhibiting growth and/or metastasis of tumor cells, comprising contacting tumor cells with an agent selected from one or more of an ionophore or a sodium channel modulator that promotes sodium efflux.

15. The method of claim 14, wherein said Na⁺ efflux does not alter the membrane potential of said cell.

16. The method of claim 14, wherein said method inhibits migration and metastasis of the tumor cell.

17. A method of promoting one or more of proliferation or differentiation, comprising administering an amount of an agent effective to increase intracellular sodium concentration in a cell, wherein said agent induces Na⁺ influx into said cell, thereby promoting one or more of proliferation or differentiation.

18. A method of promoting tissue regeneration, comprising administering an amount of an agent effective to increase intracellular sodium concentration in a cell, wherein said agent induces Na⁺ influx into said cell, thereby promoting cell proliferation to promote tissue regeneration.

19. The method of claim 18, wherein the method promotes innervation of said tissue.

20. The method of claim 18, wherein said agent induces Na⁺ influx into said cell via an endogenously expressed voltage-gated sodium channel.

21. The method of claim 20, wherein said voltage-gated sodium channel is a Na_{V}1.2 channel.

22. The method of claim 18, wherein said agent is a sodium ionophore.

23. The method of claim 22, wherein said agent is monensin.

24. The method of claim 18, wherein said agent is insulin.

25. The method of claim 18, wherein said agent is a voltage-gated sodium channel opener.

26. The method of claim 17, wherein the method further comprises administering said agent in the presence of a medium having a higher sodium concentration relative to the intracellular sodium concentration in the cell prior to administration of said agent.

27. The method of claim 17, wherein said Na⁺ influx does not alter the membrane potential of said cell.

28. The method of claim 17, wherein said cell is in a non-proliferative state prior to administration of said agent.

29. The method of claim 17, wherein said cell is a mesenchymal cell.

30. The method of claim 24, wherein said method promotes regeneration of an appendage or organ.

31. The method of claim 24, wherein said method promotes regeneration of one or more of muscle tissue and neuronal tissue.

32. The method of claim 17, wherein said cell is a progenitor cell.

33. The method of claim 32, wherein the method comprises administering said agent to a culture comprising said progenitor cell.

34. The method of claim 33, wherein said progenitor cell is selected from one or more of an embryonic stem cell, a neural progenitor cell, a neural crest progenitor cell, a mesenchymal stem cell, or a muscle progenitor cell.

35. The method of claim 34, wherein said agent is a small molecule.

36-90. (canceled)


SEQUENCE LISTING (2 sequences)

SEQ ID NO: 1 - DNA, 21 bp, Artificial Sequence
  Description of Artificial Sequence Synthetic oligonucleotide
gccatggagcattatccaatg

SEQ ID NO: 2 - DNA, 21 bp, Artificial Sequence
  Description of Artificial Sequence Synthetic oligonucleotide
gttcaagtccatctacatggc